(19) United States
(12) Patent Application Publication
(10) Pub. No.: US 2026/0007687 A1
(43) Pub. Date: Jan. 8, 2026

(54) ALPHAXALONE FAT EMULSION INJECTION AND METHOD FOR PREPARING SAME

(71) Applicant: JIANGSU NHWA PHARMACEUTICAL CO., LTD, Jiangsu (CN)

(72) Inventors: Xiangqing XU, Jiangsu (CN); Yufeng HAN, Jiangsu (CN); Weijuan PENG, Jiangsu (CN); Shuming HU, Jiangsu (CN); Chao YANG, Jiangsu (CN); Dongli WANG, Jiangsu (CN); Xi YANG, Jiangsu (CN); Ningning CHENG, Jiangsu (CN); Jianzheng CHEN, Jiangsu (CN)

(73) Assignee: JIANGSU NHWA PHARMACEUTICAL CO., LTD, Jiangsu (CN)

(21) Appl. No.: 18/993,256

(22) PCT Filed: Jul. 7, 2023
(86) PCT No.: PCT/CN2023/106281

§ 371 Date: Jan. 10, 2025

(30) Foreign Application Priority Data
Jul. 11, 2022 (CN) 202210811394.3

(51) Int. Cl. A61K 31/573; A61K 9/00; A61K 47/10; A61K 47/12; A61K 47/24; A61K 47/28; A61K 47/44

(52) U.S. Cl. CPC A61K 31/573; A61K 9/0019; A61K 47/10; A61K 47/12; A61K 47/24; A61K 47/28; A61K 47/44

(57) ABSTRACT

Provided are an alphaxalone fat emulsion injection and a method for preparing same. The alphaxalone fat emulsion injection comprises alphaxalone and a pharmaceutically acceptable salt thereof, an oil phase, an emulsifier, a co-emulsifier, an osmotic pressure regulator, a stabilizer, a pH value regulator, and water for injection.

### TECHNICAL FIELD

The present disclosure relates to the technical field of pharmaceutical formulations, and particularly relates to an alphaxalone fat emulsion injection and a preparation method therefor.

### BACKGROUND

Alphaxalone has sedative, anesthetic, anticonvulsant and neuroprotective properties by adjusting the GABA A receptor. As an effective neuroactive steroid anaesthetic, alphaxalone lacks progestogenic, estrogenic, mineralocorticoid or thymolytic activity.

Althesin is an intravenous injection composed of alphaxalone and alphadolone in a ratio of 3:1. The potency of alphadolone is only half of that of alphaxalone, but alphadolone can increase the solubility of alphaxalone by three times. The Althesin solution contains 9 mg of alphaxalone and 3 mg of alphadolone per milliliter. Althesin can achieve rapid onset and offset of the anesthetic effect, has little irritation to blood vessels, and has only slight cardiovascular and respiratory side effects.

To enhance the solubility of Althesin, the polyethoxylated castor oil excipient Cremophor EL (CAS registry number 61791-12-6) is typically added to the intravenous injection. By inducing and maintaining anesthesia, the drug was used in clinical anesthesia practice in many countries during 1972 to 1984. Since 1984, Althesin was withdrawn from the market as a human intravenous anesthetic. Despite having a high therapeutic index, Althesin occasionally causes unpredictable but severe allergic responses.

Chinese Patent CN94190450.4 discloses an alphaxalone castor oil emulsion in which Cremophor EL is a surfactant which forms a micelle in an aqueous solution when it is above the critical micelle concentration. Cremophor EL is a good encapsulating polymer, and can significantly improve the solubility of water-insoluble drugs. Because micelles disintegrate when diluted to their critical micelle concentration or less, the Cremophor EL formulation can effectively release alphaxalone and make it bioavailable for absorption by the central nervous system. Although Cremophor EL is a good solvent for dissolving neuroactive steroid anesthetics (e.g., alphaxalone), it has biological activity, the use of which has caused severe anaphylactoid hypersensitivity, hyperlipidemia, abnormal lipoprotein patterns, red blood cell aggregation, and peripheral neuropathy.

The emulsion for injection must have a very small droplet size. In order not to cause capillary clogging and embolization in blood circulation, it is required in General Chapter 0102, Chinese Pharmacopoeia, Volume IV, on emulsion injection that the average particle size should not exceed 0.5 μm.

Furthermore, although emulsions are thermodynamically unstable systems, it is desirable that emulsions be physically and chemically stable during storage. The droplet size limit defined by General Chapter 0102, Chinese Pharmacopoeia, Volume IV, is applicable to the entire specified shelf life, typically extending to 2-3 years or more for commercial pharmaceutical formulations. All true emulsions are thermodynamically unstable and may undergo processes that tend to increase droplet size over time. These include direct droplet coalescence, i.e., the collision of two droplets with each other, forming a single new droplet, and aggregation, where the droplets adhere together to form larger masses. Aggregation can in some cases be a precursor to further aggregation into larger droplets. Eventually, these processes may lead to free oil being visible on the emulsion surface or large aggregates that rise to the surface of the container, a phenomenon known as “emulsion creaming”. The initial size increase of droplets can be measured, so that the physical stability of the emulsion can be predicted at an early stage, i.e., before the formulation exhibits a macroscopic change.

In addition, the drug components may degrade. For example, lipophilic drugs will separate into an oil phase, which provides a certain degree of protection, but hydrolytic degradation may still occur at the oil-water interface. Possible chemical degradation of parenteral fat emulsions includes oxidation of unsaturated fatty acid residues present in triglycerides and lecithins and hydrolysis of phospholipids. The hydrolysis leads to the production of free fatty acids (FFA) and lysophospholipids. These degradation products lower the pH, which may further promote degradation. Thus, the pH should be controlled during the production, and the parenteral emulsion formulation may include a buffering agent to provide additional control. Any decrease in pH over a specified shelf life may indicate chemical degradation.

For example, in the case of emulsions with a stable charge, such as those in which lecithin is used as the emulsifier, a stabilized charge may vary depending on the pH. Thus, changes in pH due to chemical degradation may also accelerate physical degradation. If the emulsion is sterically stable, for example by a poly(oxyethylene) surfactant, changes in pH will generally have little effect on the stability of the emulsion. The fat emulsion injection of the present disclosure requires the use of a high-shear homogenization step to achieve a sufficiently small droplet size for sterilization and intravenous administration. The inventors have conducted extensive studies on the formulation and process of alphaxalone in order to include it in an emulsion with a sufficiently small droplet size to enable sterilization by filtration and at the same time meet the requirements of Chinese Pharmacopoeia over the entire specified formulation shelf life.

Fat overload syndrome is a syndrome characterized by elevated triacylglycerols due to the fact that the infusion speed and/or dosage of a fat emulsion exceeds the body's fat clearance ability, and is clinically manifested as: hepatosplenomegaly, jaundice, hypoproteinemia, fever, acute respiratory distress syndrome (ARDS), metabolic acidosis, thrombocytopenia, hemorrhage, disseminated intravascular coagulation (DIC), and the like. The main causes of fat overload syndrome can be attributed to two aspects: firstly, the fat clearance ability of patients is normal, and the fat emulsion is used excessively; secondly, the fat emulsion is used in a constant amount, but the fat clearance ability of patients is reduced.

The concentration of the commercial propofol is 10 mg/mL, and the potency of alphaxalone is 5 times that of propofol. Theoretically, the potency of 10 mg/mL propofol can be reached by 2 mg/mL alphaxalone. If alphaxalone can be prepared into 6 mg/mL, the intake of phospholipid and oil for injection can be reduced, which is more beneficial to some patients with obesity and hyperlipidemia.

Alphadolone has only half the potency of alphaxalone, but alphadolone can triple the solubility of alphaxalone, and the literature Mark S. Althesin-a new intravenous anaesthetic [J]. Canda. Anaesth 1973(20): 186-191 reports that alphadolone has a solubilizing-assisting effect on alphaxalone. What is provided by the present application only comprises an alphaxalone fat emulsion injection, but alphaxalone has lower solubility without an alphaxalone cosolvent. The solubility is a technical problem which needs to be solved urgently for preparing a fat emulsion injection only containing alphaxalone with high potency and high drug loading.

### SUMMARY

The present disclosure aims to solve at least one of the aforementioned technical problems to some extent or at least to provide a useful commercial alternative. Therefore, the present disclosure aims to provide an alphaxalone fat emulsion injection which has high potency, high drug loading, stable drug quality, no irritation to blood vessels, high safety of clinical medication, and good compliance.

One aspect of the present disclosure provides an alphaxalone fat emulsion injection comprising alphaxalone or a pharmaceutically acceptable salt thereof, oil for injection, an emulsifier, a coemulsifier, an osmotic pressure regulator, a stabilizer, a pH adjuster, and water for injection.

In some specific embodiments, the oil for injection is soybean oil or a mixture of soybean oil and medium-chain triglyceride.

In some specific embodiments, the weight ratio of the soybean oil to the medium-chain triglyceride in the mixture of the soybean oil and the medium-chain triglyceride is 1-5:1-5, preferably 1:1.

In some specific embodiments, optionally, the weight ratio of the alphaxalone or the pharmaceutically acceptable salt thereof to the oil for injection (based on the weight of alphaxalone) is 1-10:50-300, preferably 1-10:100-300, preferably 1-6:100-300, preferably 1-3:100-300, and preferably 1-3:100-200. In some specific embodiments, in the alphaxalone fat emulsion injection, the amount of the oil for injection accounts for 10-30%, preferably 10-20%, of the mass concentration of the fat emulsion injection.

In some specific embodiments, in the alphaxalone fat emulsion injection, the average particle size of the oil phase is 0.15 to 0.4 μm, preferably 0.15 to 0.3 μm, and more preferably 0.15 to 0.25 μm.

In some specific embodiments, in the alphaxalone fat emulsion injection, alphaxalone or a pharmaceutically acceptable salt thereof with a purity greater than 95% is used, more preferably alphaxalone or a pharmaceutically acceptable salt thereof with a purity greater than 98%.

In some specific embodiments, in the alphaxalone fat emulsion injection, the content of alphaxalone or a pharmaceutically acceptable salt thereof is 1-10 mg/mL, preferably 2-8 mg/mL, and preferably 2 to 4 mg/mL.

In some specific embodiments, in the alphaxalone fat emulsion injection, the content of the oil for injection is 100-300 mg/mL, preferably 100-200 mg/mL.

In some specific embodiments, in the alphaxalone fat emulsion injection, the content of the emulsifier is 6-15 mg/mL, preferably 9-15 mg/mL.

In some specific embodiments, in the alphaxalone fat emulsion injection, the content of the coemulsifier is 0.05-20 mg/mL, preferably 0.1-20 mg/mL, preferably 0.1-10 mg/mL, and preferably 0.5-10 mg/mL.

In some specific embodiments, in the alphaxalone fat emulsion injection, the content of the stabilizer is 0.1-0.3 mg/mL.

In some specific embodiments, in the alphaxalone fat emulsion injection, the content of the osmotic pressure regulator is 22-25 mg/mL, preferably 22.5-25 mg/mL.

In some specific embodiments, in the alphaxalone fat emulsion injection, the content of the emulsifier is 0.6%-1.5% (wt), preferably 0.9%-1.5% (wt).

In some specific embodiments, in the alphaxalone fat emulsion injection, the content of the coemulsifier is 0.05%-1.0% (wt), preferably 0.2% (wt), 0.4% (wt), 0.6% (wt), or 0.8% (wt).

In some specific embodiments, the Zeta potential of the alphaxalone fat emulsion injection is 36 to 60 mV.

In some specific embodiments, the pH of the alphaxalone fat emulsion injection is 5.0 to 8.5, preferably 6.0 to 8.5.

In some specific embodiments, the emulsifier of the alphaxalone fat emulsion injection is egg yolk lecithin, preferably at least one of egg yolk lecithin E-80, egg yolk lecithin PL-100M, and egg yolk lecithin PC-98T, and preferably a mixture of egg yolk lecithin E-80 and egg yolk lecithin PL-100M in a weight ratio of 1:2.

In some specific embodiments, the osmotic pressure regulator is glycerol.

In some specific embodiments, the coemulsifier is a cholic acid compound or a salt thereof, preferably at least one of glycocholic acid, cholic acid, taurocholic acid, glycodeoxycholic acid, taurodeoxycholic acid, chenodeoxycholic acid, glycochenodeoxycholic acid, or taurochenodeoxycholic acid, or a pharmaceutically acceptable salt thereof. In some specific embodiments, the salt of a cholic acid compound is sodium glycocholate, sodium cholate, sodium taurocholate, sodium glycodeoxycholate, sodium taurodeoxycholate, sodium chenodeoxycholate, sodium glycochenodeoxycholate, or sodium taurochenodeoxycholate.

In some specific embodiments, the emulsifier of the alphaxalone fat emulsion injection is egg yolk lecithin, preferably at least one of egg yolk lecithin E-80, egg yolk lecithin PL-100M, and egg yolk lecithin PC-98T, and preferably a mixture of egg yolk lecithin E-80 and egg yolk lecithin PL-100M in a weight ratio of 1:2, and a pH adjuster in an amount capable of adjusting the pH to 5.0-8.5, preferably 6.0-8.5; and
optionally, the osmotic pressure regulator is glycerol;
optionally, the coemulsifier is a cholic acid compound or a salt thereof;
optionally, the stabilizer is at least one selected from oleic acid and sodium oleate, preferably sodium oleate;
optionally, the pH adjuster is sodium hydroxide, and the balance of water for injection.

In some specific embodiments, the emulsifier of the alphaxalone fat emulsion injection is selected from at least one of egg yolk lecithin E-80, egg yolk lecithin PL-100M, and egg yolk lecithin PC-98T, and preferably a mixture of egg yolk lecithin E-80 and egg yolk lecithin PL-100M in a weight ratio of 1:2, and a pH adjuster in an amount capable of adjusting the pH to 6.0-8.5; and
optionally, the osmotic pressure regulator is glycerol;
optionally, the coemulsifier is a cholic acid compound or a salt thereof, preferably at least one of glycocholic acid, cholic acid, taurocholic acid, glycodeoxycholic acid, taurodeoxycholic acid, chenodeoxycholic acid, glycochenodeoxycholic acid, or taurochenodeoxycholic acid, or a pharmaceutically acceptable salt thereof. In some specific embodiments, the salt of a cholic acid compound is sodium glycocholate, sodium cholate, sodium taurocholate, sodium glycodeoxycholate, sodium taurodeoxycholate, sodium chenodeoxycholate, sodium glycochenodeoxycholate, or sodium taurochenodeoxycholate;
optionally, the stabilizer is at least one selected from oleic acid and sodium oleate, preferably sodium oleate;
optionally, the pH adjuster is sodium hydroxide, and the balance of water for injection.

In some specific embodiments, the alphaxalone fat emulsion injection comprises 0.05%-1.0% (w/v), preferably 0.2% (w/v), 0.4% (w/v), 0.6% (w/v), or 0.8% (w/v) of the coemulsifier.

In some specific embodiments, the alphaxalone fat emulsion injection comprises 0.05%-1.0% (w/v), preferably 0.2% (w/v), 0.4% (w/v), 0.6% (w/v), or 0.8% (w/v) of the cholic acid compound or the salt thereof.

In some specific embodiments, the alphaxalone fat emulsion injection comprises 0.6%-1.5% (w/v), preferably 0.6% (w/v), 0.9% (w/v), 1.2% (w/v), or 1.5% (w/v) of the emulsifier, and further preferably 0.9% (w/v), 1.2% (w/v), or 1.5% (w/v) of the emulsifier.

In some specific embodiments, the alphaxalone fat emulsion injection comprises 0.05%-1.0% (w/v), preferably 0.2% (w/v), 0.4% (w/v), 0.6% (w/v), or 0.8% (w/v) of the sodium glycocholate.

In some specific embodiments, the alphaxalone fat emulsion injection comprises 5%-15% (w/v), preferably 5% (w/v), 10% (w/v), or 15% (w/v) of the soybean oil.

In some specific embodiments, the alphaxalone fat emulsion injection comprises 5%-15% (w/v), preferably 5% (w/v), 10% (w/v), or 15% (w/v) of the medium-chain triglyceride.

In some specific embodiments, the alphaxalone fat emulsion injection comprises 0.01%-0.03% (w/v), preferably 0.01% (w/v) or 0.03% (w/v) of the sodium oleate.

In some specific embodiments, the alphaxalone fat emulsion injection comprises 2.25%-2.5% (w/v), preferably 2.25% (w/v) or 2.5% (w/v) of the glycerol.

In some specific embodiments, the alphaxalone fat emulsion injection comprises 0.6%-1.5% (w/v), preferably 0.6% (w/v), 0.9% (w/v), 1.2% (w/v), or 1.5% (w/v) of the egg yolk lecithin, and further preferably 0.9% (w/v), 1.2% (w/v), or 1.5% (w/v) of the egg yolk lecithin.

In some embodiments, the alphaxalone fat emulsion injection comprises a cholic acid compound or a salt thereof as a coemulsifier, wherein the alphaxalone fat emulsion injection comprises 0.05%-1.0% (w/v), preferably 0.2% (w/v), 0.4% (w/v), 0.6% (w/v), or 0.8% (w/v) of the sodium glycocholate; and in the alphaxalone fat emulsion injection, the average particle size of the oil phase is 0.15 to 0.4 μm, preferably 0.15 to 0.3 μm, and more preferably 0.15 to 0.25 μm. Furthermore, in the alphaxalone fat emulsion injection, the content of alphaxalone or the pharmaceutically acceptable salt thereof is 2 to 4 mg/mL. Furthermore, the alphaxalone fat emulsion injection comprises 0.6-1.5% (wt), preferably 0.9-1.5% (wt) of the egg yolk lecithin as an emulsifier. Furthermore, the Zeta potential of the alphaxalone fat emulsion injection is 36 to 60 m V. Furthermore, the pH of the alphaxalone fat emulsion injection is 5.0 to 8.5, preferably 6.0 to 8.5.

In some specific embodiments, the alphaxalone fat emulsion injection has a pH of 5.0-8.5; the components include the following in parts by weight: 1-10 of alphaxalone; 50-200 of soybean oil; 0-100 of medium-chain triglyceride; 9-15 of emulsifier; 0.1-20 of coemulsifier; 0.1-0.3 of stabilizer; and 22-25 of glycerol.

In some specific embodiments, the alphaxalone fat emulsion injection has a pH of 6.0-8.5; the components include the following in parts by weight: 1-10 of alphaxalone; 50-200 of soybean oil; 0-100 of medium-chain triglyceride; 9-15 of emulsifier; 0.1-20 of coemulsifier; 0.1-0.3 of stabilizer; and 22-25 of glycerol.

In some specific embodiments, the alphaxalone fat emulsion injection has a pH of 6.0-8.5; the components include the following in parts by weight: 2-8 of alphaxalone; 50-200 of soybean oil; 0-100 of medium-chain triglyceride; 9-12 of emulsifier; 0.1-10 of coemulsifier; 0.1-0.3 of stabilizer; and 22-25 of glycerol.

In some specific embodiments, the alphaxalone fat emulsion injection has a pH of 6.0-8.5, and the alphaxalone fat emulsion injection comprises per 1 mL: 1-10 mg of alphaxalone; 50-100 mg of soybean oil; 50-100 mg of medium-chain triglyceride; 9-15 mg of egg yolk lecithin; 0.1-10 mg of sodium glycocholate; 0.1-0.3 mg of sodium oleate; sodium hydroxide in an amount capable of controlling the pH of the injection to 6.0-8.5; and the balance of water for injection.

In some specific embodiments, the alphaxalone fat emulsion injection has a pH of 6.0-8.5, and the alphaxalone fat emulsion injection comprises per 1 mL: 1-10 mg of alphaxalone; 100-200 mg of soybean oil; 9-15 mg of egg yolk lecithin; 0.1-10 mg of sodium glycocholate; 0.1-0.3 mg of sodium oleate; sodium hydroxide in an amount capable of controlling the pH of the injection to 6.0-8.5; and the balance of water for injection.

In some specific embodiments, the alphaxalone fat emulsion injection has a pH of 6.0-8.5, and the alphaxalone fat emulsion injection comprises per 1 mL: 1-10 mg of alphaxalone; 50-100 mg of soybean oil; 50-100 mg of medium-chain triglyceride; 9-15 mg of egg yolk lecithin; 0.1-10 mg of sodium glycocholate; 0.1-0.3 mg of sodium oleate; and 25 mg of glycerol.

In some specific embodiments, the alphaxalone fat emulsion injection has a pH of 6.0-8.5, and the alphaxalone fat emulsion injection comprises per 1 mL: 1-10 mg of alphaxalone; 100-200 mg of soybean oil; 9-15 mg of egg yolk lecithin; 0.1-10 mg of sodium glycocholate; 0.1-0.3 mg of sodium oleate; 25 mg of glycerol; sodium hydroxide in an amount capable of controlling the pH of the injection to 6.0-8.5; and the balance of water for injection.

In some specific embodiments, the alphaxalone fat emulsion injection has a pH of 6.0-8.5, and the alphaxalone fat emulsion injection comprises per 1 mL: 1-10 mg of alphaxalone; 100 mg of soybean oil; 100 mg of medium-chain triglyceride; 9-15 mg of egg yolk lecithin; 0.1-10 mg of glycocholic acid; 0.1-0.3 mg of sodium oleate; 25 mg of glycerol; sodium hydroxide in an amount capable of controlling the pH of the injection to 6.0-8.5; and the balance of water for injection;
or the pH is 6.0-8.5, and the alphaxalone fat emulsion injection comprises per 1 mL: 1-10 mg of alphaxalone; 200 mg of soybean oil; 9-15 mg of egg yolk lecithin; 0.1-10 mg of cholic acid; 0.1-0.3 mg of sodium oleate; 25 mg of glycerol; sodium hydroxide in an amount capable of controlling the pH of the injection to 6.0-8.5; and the balance of water for injection.

In some specific embodiments, the alphaxalone fat emulsion injection has a pH of 6.0-8.5, and the alphaxalone fat emulsion injection comprises per 1 mL: 2-8 mg of alphaxalone; 100 mg of soybean oil; 100 mg of medium-chain triglyceride; 9-12 mg of egg yolk lecithin; 0.1-10 mg of sodium glycocholate; 0.1-0.3 mg of sodium oleate; 25 mg of glycerol; sodium hydroxide in an amount capable of controlling the pH of the injection to 6.0-8.5; and the balance of water for injection;
or the pH is 6.0-8.5, and the alphaxalone fat emulsion injection comprises per 1 mL: 2-8 mg of alphaxalone; 200 mg of soybean oil; 9-12 mg of egg yolk lecithin; 0.1-10 mg of sodium glycocholate; 0.1-0.3 mg of sodium oleate; 25 mg of glycerol; sodium hydroxide in an amount capable of controlling the pH of the injection to 6.0-8.5; and the balance of water for injection;
or the pH is 6.0-8.5, and the alphaxalone fat emulsion injection comprises per 1 mL: 2-8 mg of alphaxalone; 100 mg of soybean oil; 50 mg of medium-chain triglyceride; 9-12 mg of egg yolk lecithin; 0.1-20 mg of sodium glycocholate; 0.1-0.3 mg of sodium oleate; 25 mg of glycerol; sodium hydroxide in an amount capable of controlling the pH of the injection to 6.0-8.5; and the balance of water for injection;
or the pH is 6.0-8.5, and the alphaxalone fat emulsion injection comprises per 1 mL: 2-8 mg of alphaxalone; 100 mg of soybean oil; 50 mg of medium-chain triglyceride; 9-12 mg of egg yolk lecithin; 0.1-10 mg of sodium glycocholate; 0.1-0.3 mg of sodium oleate; 25 mg of glycerol; sodium hydroxide in an amount capable of controlling the pH of the injection to 6.0-8.5; and the balance of water for injection;
or the pH is 6.0-8.5, and the alphaxalone fat emulsion injection comprises per 1 mL: 2-8 mg of alphaxalone; 50 mg of soybean oil; 50 mg of medium-chain triglyceride; 9-12 mg of egg yolk lecithin; 0.1-10 mg of sodium glycocholate; 0.1-0.3 mg of sodium oleate; 25 mg of glycerol; sodium hydroxide in an amount capable of controlling the pH of the injection to 6.0-8.5; and the balance of water for injection.

In some specific embodiments, the alphaxalone fat emulsion injection has a pH of 6.0-8.5, and the alphaxalone fat emulsion injection comprises per 1 mL: 1-10 mg of alphaxalone; 50-200 mg of soybean oil; 50-100 mg of medium-chain triglyceride; 9-15 mg of egg yolk lecithin; 0.1-20 mg of sodium glycocholate; 0.1-0.3 mg of sodium oleate; 22.5-25 mg of glycerol; sodium hydroxide in an amount capable of controlling the pH of the injection to 6.0-8.5; and the balance of water for injection.

In some specific embodiments, the alphaxalone fat emulsion injection has a pH of 6.0-8.5, and the alphaxalone fat emulsion injection comprises per 1 mL: 1-10 mg of alphaxalone; 50-200 mg of soybean oil; 50-100 mg of medium-chain triglyceride; 9-15 mg of egg yolk lecithin; 0.1-10 mg of sodium glycocholate; 0.1-0.3 mg of sodium oleate; 22.5-25 mg of glycerol; sodium hydroxide in an amount capable of controlling the pH of the injection to 6.0-8.5; and the balance of water for injection;
or the pH is 8.0, and the alphaxalone fat emulsion injection comprises per 1 mL: 2 mg of alphaxalone; 50 mg of soybean oil; 50 mg of medium-chain triglyceride; 4 mg of egg yolk lecithin E-80; 8 mg of egg yolk lecithin PL-100M; 3 mg of sodium glycocholate; 0.3 mg of sodium oleate; 25 mg of glycerol; sodium hydroxide in an amount capable of controlling the pH of the injection to 8.0; and the balance of water for injection;
or the pH is 6.0, and the alphaxalone fat emulsion injection comprises per 1 mL: 3 mg of alphaxalone; 100 mg of soybean oil; 100 mg of medium-chain triglyceride; 12 mg of egg yolk lecithin E-80; 2 mg of sodium glycocholate; 0.3 mg of sodium oleate; 25 mg of glycerol; sodium hydroxide in an amount capable of controlling the pH of the injection to 6.0; and the balance of water for injection;
or the pH is 8.0, and the alphaxalone fat emulsion injection comprises per 1 mL: 4 mg of alphaxalone; 50 mg of soybean oil; 50 mg of medium-chain triglyceride; 9 mg of egg yolk lecithin E-80; 4 mg of sodium glycocholate; 0.3 mg of sodium oleate; 25 mg of glycerol; sodium hydroxide in an amount capable of controlling the pH of the injection to 8.0; and the balance of water for injection;

or the pH is 8.0, and the alphaxalone fat emulsion injection comprises per 1 mL: 4 mg of alphaxalone; 200 mg of soybean oil; 12 mg of egg yolk lecithin E-80; 4 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection comprises per 1 mL: 4 mg of alphaxalone; 200 mg of soybean oil; 12 mg of egg yolk lecithin E-80; 1 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection comprises per 1 mL: 2 mg of alphaxalone; 100 mg of soybean oil; 12 mg of egg yolk lecithin E-80; 4 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection comprises per 1 mL: 4 mg of alphaxalone; 100 mg of soybean oil; 100 mg of medium-chain triglyceride; 15 mg of egg yolk lecithin E-80; 3 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection comprises per 1 mL: 4 mg of alphaxalone; 100 mg of soybean oil; 100 mg of medium-chain triglyceride; 12 mg of egg yolk lecithin E-80; 1 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection comprises per 1 mL: 4 mg of alphaxalone; 100 mg of soybean oil; 100 mg of medium-chain triglyceride; 15 mg of egg yolk lecithin E-80; 3 mg of sodium glycocholate; 0.3 mg of sodium oleate; 25 mg of glycerol; sodium hydroxide in an amount capable of controlling the pH of the injection to 8.0; and the balance of water for injection;
or the pH is 7.0, and the alphaxalone fat emulsion injection comprises per 1 mL: 5 mg of alphaxalone; 100 mg of soybean oil; 100 mg of medium-chain triglyceride; 15 mg of egg yolk lecithin E-80; 3 mg of sodium glycocholate; 0.3 mg of sodium oleate; 25 mg of glycerol; sodium hydroxide in an amount capable of controlling the pH of the injection to 7.0; and the balance of water for injection;
or the pH is 7.0, and the alphaxalone fat emulsion injection comprises per 1 mL: 5 mg of alphaxalone; 100 mg of soybean oil; 100 mg of medium-chain triglyceride; 12 mg of egg yolk lecithin E-80; 1 mg of sodium glycocholate; 0.3 mg of sodium oleate; 25 mg of glycerol; sodium hydroxide in an amount capable of controlling the pH of the injection to 7.0; and the balance of water for injection;
or the pH is 7.5, and the alphaxalone fat emulsion injection comprises per 1 mL: 6 mg of alphaxalone; 50 mg of soybean oil; 50 mg of medium-chain triglyceride; 12 mg of egg yolk lecithin E-80; 2 mg of sodium glycocholate; 0.3 mg of sodium oleate; 25 mg of glycerol; sodium hydroxide in an amount capable of controlling the pH of the injection to 7.5; and the balance of water for injection;
or the pH is 8.0, and the alphaxalone fat emulsion injection comprises per 1 mL: 7 mg of alphaxalone; 100 mg of soybean oil; 100 mg of medium-chain triglyceride; 12 mg of egg yolk lecithin; 4 mg of sodium glycocholate; 0.3 mg of sodium oleate; 25 mg of glycerol; sodium hydroxide in an amount capable of controlling the pH of the injection to 8.0; and the balance of water for injection;
or the pH is 7.5, and the alphaxalone fat emulsion injection comprises per 1 mL: 7 mg of alphaxalone; 100 mg of soybean oil; 100 mg of medium-chain triglyceride; 12 mg of egg yolk lecithin; 1 mg of sodium glycocholate; 0.3 mg of sodium oleate; 25 mg of glycerol; sodium hydroxide in an amount capable of controlling the pH of the injection to 7.5; and the balance of water for injection.

In some specific embodiments, the alphaxalone fat emulsion injection has a pH of 6.0-8.5, and the alphaxalone fat emulsion injection consists of per 1 mL: 1-10 mg of alphaxalone; 50-200 mg of soybean oil; 50-100 mg of medium-chain triglyceride; 9-15 mg of egg yolk lecithin; 0.1-10 mg of sodium glycocholate; 0.1-0.3 mg of sodium oleate; 22.5-25 mg of glycerol; sodium hydroxide in an amount capable of controlling the pH of the injection to 6.0-8.5; and the balance of water for injection;
or the pH is 8.0, and the alphaxalone fat emulsion injection consists of per 1 mL: 2 mg of alphaxalone; 50 mg of soybean oil; 50 mg of medium-chain triglyceride; 4 mg of egg yolk lecithin E-80; 8 mg of egg yolk lecithin PL-100M; 3 mg of sodium glycocholate; 0.3 mg of sodium oleate; 25 mg of glycerol; sodium hydroxide in an amount capable of controlling the pH of the injection to 8.0; and the balance of water for injection;
or the pH is 6.0, and the alphaxalone fat emulsion injection consists of per 1 mL: 3 mg of alphaxalone; 100 mg of soybean oil; 100 mg of medium-chain triglyceride; 12 mg of egg yolk lecithin E-80; 2 mg of sodium glycocholate; 0.3 mg of sodium oleate; 25 mg of glycerol; sodium hydroxide in an amount capable of controlling the pH of the injection to 6.0; and the balance of water for injection;
or the pH is 8.0, and the alphaxalone fat emulsion injection consists of per 1 mL: 4 mg of alphaxalone; 50 mg of soybean oil; 50 mg of medium-chain triglyceride; 9 mg of egg yolk lecithin E-80; 4 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection consists of per 1 mL: 4 mg of alphaxalone; 200 mg of soybean oil; 12 mg of egg yolk lecithin E-80; 4 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection consists of per 1 mL: 4 mg of alphaxalone; 200 mg of soybean oil; 12 mg of egg yolk lecithin E-80; 1 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection consists of per 1 mL: 2 mg of alphaxalone; 100 mg of soybean oil; 12 mg of egg yolk lecithin E-80; 4 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection consists of per 1 mL: 4 mg of alphaxalone; 100 mg of soybean oil; 100 mg of medium-chain triglyceride; 15 mg of egg yolk lecithin E-80; 3 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection consists of per 1 mL: 4 mg of alphaxalone; 100 mg of soybean oil; 100 mg of medium-chain triglyceride; 12 mg of egg yolk lecithin E-80; 1 mg of sodium glycocholate; 0.3 mg of sodium oleate; 25 mg of glycerol; sodium hydroxide in an amount capable of controlling the pH of the injection to 8.0; and the balance of water for injection;
or the pH is 8.0, and the alphaxalone fat emulsion injection consists of per 1 mL: 4 mg of alphaxalone; 100 mg of soybean oil; 100 mg of medium-chain triglyceride; 15 mg of egg yolk lecithin E-80; 3 mg of sodium glycocholate; 0.3 mg of sodium oleate; 25 mg of glycerol; sodium hydroxide in an amount capable of controlling the pH of the injection to 8.0; and the balance of water for injection;
or the pH is 7.0, and the alphaxalone fat emulsion injection consists of per 1 mL: 5 mg of alphaxalone; 100 mg of soybean oil; 100 mg of medium-chain triglyceride; 15 mg of egg yolk lecithin E-80; 3 mg of sodium glycocholate; 0.3 mg of sodium oleate; 25 mg of glycerol; sodium hydroxide in an amount capable of controlling the pH of the injection to 7.0; and the balance of water for injection;
or the pH is 7.0, and the alphaxalone fat emulsion injection consists of per 1 mL: 5 mg of alphaxalone; 100 mg of soybean oil; 100 mg of medium-chain triglyceride; 12 mg of egg yolk lecithin E-80; 1 mg of sodium glycocholate; 0.3 mg of sodium oleate; 25 mg of glycerol; sodium hydroxide in an amount capable of controlling the pH of the injection to 7.0; and the balance of water for injection;
or the pH is 7.5, and the alphaxalone fat emulsion injection consists of per 1 mL: 6 mg of alphaxalone; 50 mg of soybean oil; 50 mg of medium-chain triglyceride; 12 mg of egg yolk lecithin E-80; 2 mg of sodium glycocholate; 0.3 mg of sodium oleate; 25 mg of glycerol; sodium hydroxide in an amount capable of controlling the pH of the injection to 7.5; and the balance of water for injection;
or the pH is 8.0, and the alphaxalone fat emulsion injection consists of per 1 mL: 7 mg of alphaxalone; 100 mg of soybean oil; 100 mg of medium-chain triglyceride; 12 mg of egg yolk lecithin; 4 mg of sodium glycocholate; 0.3 mg of sodium oleate; 25 mg of glycerol; sodium hydroxide in an amount capable of controlling the pH of the injection to 8.0; and the balance of water for injection;
or the pH is 7.5, and the alphaxalone fat emulsion injection consists of per 1 mL: 7 mg of alphaxalone; 100 mg of soybean oil; 100 mg of medium-chain triglyceride; 12 mg of egg yolk lecithin; 1 mg of sodium glycocholate; 0.3 mg of sodium oleate; 25 mg of glycerol; sodium hydroxide in an amount capable of controlling the pH of the injection to 7.5; and the balance of water for injection.

In another aspect of the present disclosure, provided is a method for preparing an alphaxalone fat emulsion injection, comprising:
(1) heating oil for injection to 50-75° C., then adding an emulsifier and alphaxalone, mixing, and shearing until completely dissolved and uniformly dispersed so as to obtain a first mixture containing an alphaxalone oil phase;
preferably, the oil for injection is soybean oil or a mixture of soybean oil and medium-chain triglyceride;
more preferably, the oil for injection is a mixture of soybean oil and medium-chain triglyceride in a weight ratio of 1:1;
preferably, the emulsifier is egg yolk lecithin; more preferably, the emulsifier is a mixture of egg yolk lecithin PL-100M and egg yolk lecithin E-80;
(2) heating water for injection to 50-75° C., then adding a coemulsifier, a stabilizer, and an osmotic pressure regulator, and stirring uniformly so as to obtain a second mixture forming an aqueous phase; preferably, the coemulsifier is sodium glycocholate, the stabilizer is sodium oleate, and the osmotic pressure regulator is glycerol;
(3) mixing the first mixture and the second mixture and shearing during the mixing process so as to obtain a third mixture forming primary emulsion;
(4) adjusting the pH of the third mixture to 6.5-11.5, preferably 6.5-10.5, and preferably 6.5-10, using a pH adjuster so as to obtain a fourth mixture, and performing low-pressure homogenization once to obtain a fifth mixture; preferably, the pH adjuster is sodium hydroxide;
(5) homogenizing the fifth mixture at high pressure for 3 times or more, preferably 4-8 times, and more preferably 5-6 times, with nitrogen introduced below the liquid surface to saturation during homogenization so as to obtain a sixth mixture;
(6) filling the sixth mixture into a container, introducing nitrogen for protection, and performing wet-heat sterilization so as to obtain an alphaxalone fat emulsion injection with a pH of 6.0-8.5.

In another aspect of the present disclosure, provided is a method for preparing an alphaxalone fat emulsion injection, comprising:
(1) heating oil for injection to 50-75° C., then adding an emulsifier and alphaxalone, mixing, and shearing until completely dissolved and uniformly dispersed so as to obtain a first mixture containing an alphaxalone oil phase;
preferably, the oil for injection is soybean oil or a mixture of soybean oil and medium-chain triglyceride; more preferably, the oil for injection is a mixture of soybean oil and medium-chain triglyceride in a weight ratio of 1:1;
preferably, the emulsifier is egg yolk lecithin; more preferably, the emulsifier is a mixture of egg yolk lecithin PL-100M and egg yolk lecithin E-80;
(2) heating water for injection to 50-75° C., then adding a coemulsifier, a stabilizer, an osmotic pressure regulator, and a pH adjuster, and stirring until uniformly mixed so as to obtain a second mixture forming an aqueous phase; the pH is 7-11.5, preferably 7-10.5, and preferably 7-10; preferably, the coemulsifier is sodium glycocholate, the stabilizer is sodium oleate, the osmotic pressure regulator is glycerol, and the pH adjuster is sodium hydroxide;
(3) mixing the first mixture and the second mixture and shearing during the mixing process so as to obtain a third mixture forming primary emulsion;
(4) optionally, adjusting the pH of the third mixture to 6.5-11.5, preferably 6.5-10.5, and preferably 6.5-10, using a pH adjuster so as to obtain a fourth mixture; preferably, the pH adjuster is sodium hydroxide;
(5) homogenizing the mixture obtained in (3) or (4) at low pressure once to obtain a fifth mixture; homogenizing the fifth mixture at high pressure for 3 times or more, preferably 4-8 times, and more preferably 5-6 times, with nitrogen introduced below the liquid surface to saturation during homogenization so as to obtain a sixth mixture;
(6) filling the sixth mixture into a container, introducing nitrogen for protection, and performing wet-heat sterilization so as to obtain an alphaxalone fat emulsion injection with a pH of 6.0-8.5.

In some specific embodiments, the shearing and mixing are performed at a rotation speed of 5000-16000 rpm for 5-25 min; preferably, the rotation speed of the shearing during the mixing process is 10000 rpm, and the rotation speed of the shearing after the completion of mixing is 15000-16000 rpm. In some specific embodiments, in step (3) of the preparation method for the alphaxalone fat emulsion, the shearing and mixing are performed at a rotation speed of 5000-16000 rpm for 5-25 min.

In some specific embodiments, the low-pressure homogenization in step (4) in the preparation method for the alphaxalone fat emulsion is performed at 100/160 bar, the high-pressure homogenization in step (5) is performed at 500-1000 bar for 5-8 times, and the high-pressure homogenization temperature is controlled at 30-70° C.

### Terminology

The term “isotonic” means that it has an osmotic pressure equal to or similar to that of physiological body fluids. Body fluids typically have an osmotic pressure often described as corresponding to 0.9% (weight/volume) aqueous sodium chloride solution. In the present application, glycerol is used to adjust the isotonic property. For the amount of glycerol, every 1 mL of the alphaxalone fat emulsion injection comprises 22-25 mg, specifically 22 mg/mL, 22.5 mg/mL, 23 mg/mL, 23.5 mg/mL, 24 mg/mL, 24.5 mg/mL, and specifically 25 mg/mL of glycerol.

The term “medium-chain oil” means medium-chain triglycerides; the term “long-chain oil” means soybean oil.

The term “w/v” refers to the mass content of a component per unit volume of injection solution in unit “g/mL”.

The term “coemulsifier” has a positive effect on the stability of an emulsion in a general emulsion system.

The coemulsifier of the present application is specifically a cholic acid compound or a salt thereof, and more specifically glycocholic acid and sodium glycocholate, cholic acid, taurocholic acid, glycodeoxycholic acid or taurodeoxycholic acid, chenodeoxycholic acid, glycochenodeoxycholic acid or taurochenodeoxycholic acid, etc., which can improve the solubility of the drug, and improve the stability of the fat emulsion and the drug, etc.

The term “AFSL” is alphaxalone.

In this specification, “about” may be understood as being within 10%, 9%, 8%, 7%, 6%, 5%, 4%, 3%, 2%, 1%, 0.5%, 0.1%, 0.05%, or 0.01% of the value. All values provided herein are modified by the term “about” unless otherwise specifically stated.

### Technical Effects

The solubility of alphaxalone in castor oil is higher than that in medium-chain oil and long-chain oil. The present disclosure discovers the optimal ratio of medium-chain oil and long-chain oil for injection with poor solubility to alphaxalone by exploring the optimal ratio of oil for injection, and the content of the oil for injection should be in a certain appropriate range, which can ensure that the emulsifier can completely emulsify the oil phase and the aqueous phase.

Compared with the present disclosure, the formulation without adding sodium glycocholate can only satisfy the preparation in small strength (2 mg/mL). For the preparation in large strength (4 mg/mL), the starting material precipitates after being placed for 10 days under a refrigeration condition, oil drops exist on the surface of the fat emulsion, the phenomenon of demulsification and the like occurs, and the stability is poor. The sodium glycocholate and lecithin form a synergistic effect to improve the dissolution effect. The average particle size obtained by preparation is lower than that of the group without adding sodium glycocholate, the uniformity of the particle size is good, the pH is confirmed by stability tests to be stable, and no obvious reduction occurs, which shows that the emulsion has good stability, is beneficial to storage, has no harsh requirements on storage conditions, and shows that the sodium glycocholate or glycocholic acid has the functions of dissolution aiding and stabilization, can increase the affinity of a medicament and an oil phase, and thus improves the stability of the formulation. Compared with castor oil formulation, the formulation has smaller particle size, is more stable, and significantly reduces the occurrence of allergy, which is safer.

In addition, compared with a formulation without adding glycocholic acid or sodium glycocholate, the drug loading can be significantly improved from 2 mg/mL to 6 mg/mL, which is improved by three times.

Pharmacological experiments prove that the fat emulsion provided by the present application has sensitization significantly lower than that of the emulsion containing castor oil prepared according to CN94190450.4, has average particle size significantly smaller than that of the castor oil emulsion group, has encapsulation efficiency higher than that of the castor oil formulation group, has injection pain smaller than that of the castor oil emulsion group, and has various indexes of stability better than those of the castor oil emulsion group.

The alphaxalone fat emulsion injection described herein avoids the use of excipients such as castor oil, cyclodextrin, and the like which are easy to cause adverse reactions. The present disclosure uses an emulsion prepared by a new formula, and uses soybean oil and medium-chain triglyceride in a weight ratio of 1:1 in the formulation as oil for injection, sodium oleate as a stabilizer, egg yolk lecithin E-80 as an emulsifier, and sodium glycocholate or glycocholic acid as a coemulsifier. The quality of the prepared alphaxalone fat emulsion injection is more stable. The use of sodium glycocholate or glycocholic acid as a coemulsifier greatly improves the stability of the alphaxalone fat emulsion injection, can prevent unstable conditions of emulsion drop aggregation, combination and the like of fat emulsion caused by the change of storage conditions in the placement of the alphaxalone formulation, avoids irritation to blood vessels, and improves the safety and the compliance of clinical administration.

### BRIEF DESCRIPTION OF THE DRAWING

FIG. 1 is a stratification diagram of primary emulsion of 1.2% sodium glycocholate fat emulsion.

FIG. 2 is a stratification diagram of primary emulsion of 1.4% sodium glycocholate fat emulsion.

FIG. 3 is a stratification diagram of primary emulsion of 2.0% sodium glycocholate fat emulsion.

FIG. 4 shows API precipitation on day 20 of the sterilized sample without the addition of sodium glycocholate at 4 mg/mL.

FIG. 5 shows the measurement of the plasma histamine content in mice after administration by intravenous injection.

FIG. 6 shows a rat myoelectricity stimulation test.

### DETAILED DESCRIPTION

Egg yolk lecithin: E80, purchased from Lipoid GmbH, Germany; glycerol, purchased from Shantou Jiahe Biotech Co., Ltd.; sodium glycocholate, purchased from Hebei Zhentian Food Additive Co., Ltd.; soybean oil, purchased from Guangzhou Baiyunshan Hanfang Modern Pharmaceutical Co., Ltd.; medium-chain triglyceride (57.1% caprylic acid, 42.8% capric acid), purchased from Liaoning Xinxing Pharmaceutical Co., Ltd.; sodium oleate, purchased from Lipoid GmbH, Germany; alphaxalone, self-prepared by Jiangsu Nhwa Pharmaceutical Co., Ltd. with the purity of 95% or more; propofol (manufacturer: Jiangsu Nhwa Pharmaceutical Co., Ltd., batch No. BB200215); Cremophor EL (manufacturer: Sinopharm Chemical Reagent Co., Ltd., batch No. 20220104); sulfobutyl-β-cyclodextrin/SBE-β-CD (manufacturer: Zibo Qianhui Biotech Co., Ltd., batch No. SB210911).

### Examples 1-4 Preparation Method of Emulsions without Adding Sodium Glycocholate (Batch Size 1000 mL)

Preparation of 0.1 mol/L sodium hydroxide: 0.4 g of sodium hydroxide was weighed out, dissolved in a proper amount of water, cooled and transferred to a 100 mL volumetric flask, and the volume was brought to the mark for later use.

Preparation of oil phase: 100 g of soybean oil and 100 g of medium-chain triglyceride were added to a 500 mL beaker and heated in a 70° C. water bath. Egg yolk lecithin and alphaxalone (AFSL) were weighed out according to the formulation amount, added to the oil phase, and sheared at 10000 rpm for 5 min to obtain the oil phase.

Preparation of aqueous phase: a proper amount of water for injection was weighed out and added to a 2000 mL beaker, warmed in a water bath with the water temperature controlled at 70° C.; 0.3 g of sodium oleate and 25 g of glycerol were added to the aqueous phase and stirred uniformly using a glass rod to obtain the aqueous phase.

Preparation of primary emulsion: the oil phase was added to the aqueous phase while shearing (10000 rpm), and the temperature of the primary emulsion was controlled at 70° C.; after the oil phase was added, the shearing speed was adjusted to 15400 rpm, shearing was performed for 5 min, and the pH of the primary emulsion was adjusted to 9.0 with 0.1 mol/L sodium hydroxide to obtain the primary emulsion.

Preparation of product: low-pressure homogenization pressure: 100 bar/160 bar, homogenization was performed once. High-pressure homogenization pressure: 100 bar/600 bar, homogenization was performed six times. After homogenization, the sample was sealed, filled with nitrogen, and sterilized (121° C., 15 min).

### 1.1 Formulation Composition

| Formulation | | | | | | | | | |
|---|---|---|---|---|---|---|---|---|---|
| composition | Example 1 | | Example 2 | | Example 3 | | Example 4 | | Use |
| Alphaxalone | 2 | g | 3 | g | 4 | g | 5 | g | Active |
| | | | | | | | | | ingredient |
| Soybean oil | 100 | g | 100 | g | 100 | g | 100 | g | Oil phase |
| (for injection) | | | | | | | | | |
| Medium-chain | 100 | g | 100 | g | 100 | g | 100 | g | Oil phase |
| triglyceride | | | | | | | | | |
| Egg yolk lecithin | 12 | g | 12 | g | 12 | g | 12 | g | Emulsifier |
| Glycerol | 25 | g | 25 | g | 25 | g | 25 | g | Osmotic |
| | | | | | | | | | pressure |
| | | | | | | | | | regulator |
| Sodium oleate | 0.3 | g | 0.3 | g | 0.3 | g | 0.3 | g | Stabilizer |

| | | | | | |
|---|---|---|---|---|---|
| Sodium hydroxide | Proper | Proper | Proper | Proper | pH adjuster |
| | amount | amount | amount | amount | |
| Water for injection | Proper | Proper | Proper | Proper | Aqueous phase |
| | amount | amount | amount | amount | |

### 1.2. Experimental Results

| Item | Example 1 | Example 2 | Example 3 | Example 4 |
|---|---|---|---|---|
| Appearance of oil phase | Clear | Clear | Clear | Clear |
| Appearance of product | Uniform milky | Uniform milky | Uniform milky | Uniform milky |
| | white | white | white | white |
| Content (i.e., content of | 102.35 | 102.68 | 100.19 | 94.16 |
| active pharmaceutical | | | | |
| ingredient in emulsion | | | | |
| product, %) | | | | |
| Encapsulation efficiency | 85.76 | 88.46 | 87.80 | 90.56 |
| (%) | | | | |
| pH | 7.70 | 7.58 | 7.55 | 7.60 |
| Particle size (μm) | 0.262 | 0.297 | 0.227 | 0.238 |
| Zeta potential (mV) | 33.3 | 34.9 | 32.1 | 39.5 |

1.3 Conclusion: the active pharmaceutical ingredient of the 4 strength samples were all completely dissolved in the oil phase, and the solution was clear and transparent; the appearance, encapsulation efficiency, pH, particle size, and Zeta potential of the product of the 4 batches met the requirements immediately after sterilization, but the content tended to decrease with the increase of the strength, and particularly, the content of Example 4 (5 mg/mL) was only 94.16% immediately after sterilization.

### 1.4 Experiments of Influencing Factors

Samples of 2 mg/mL, 3 mg/mL, 4 mg/mL, and 5 mg/mL strengths were stored under high temperature (60° C., 40° C.) and low temperature (4° C.). Sampling and testing were conducted on day 5, day 10, and day 30. The results are shown in the table below.

### Example 1: (Strength: 2 mg/mL)

| | | | | | Particle | Zeta |
|---|---|---|---|---|---|---|
| | | Content | Encapsulation | | size | potential |
| | Appearance | % | efficiency % | pH | (μm) | (mV) |
| After sterilization | Uniform | 102.35 | 85.76 | 7.70 | 0.262 | 33.3 |
| | milky white | | | | | |
| Low temperature | Uniform | 101.37 | 87.12 | 7.75 | 0.252 | 38.5 |
| 4° C.-5 d | milky white | | | | | |
| High temperature | Uniform | 102.19 | 86.12 | 7.65 | 0.252 | 39.8 |
| 40° C.-5 d | milky white | | | | | |
| High temperature | Uniform | 102.78 | 87.72 | 7.34 | 0.255 | 41.2 |
| 60° C.-5 d | milky white | | | | | |
| Low temperature | Uniform | 102.73 | 87.23 | 7.70 | 0.267 | 33.9 |
| 4° C.-10 d | milky white | | | | | |
| High temperature | Uniform | 101.86 | 86.42 | 7.27 | 0.256 | 37.1 |
| 40° C.-10 d | milky white | | | | | |
| High temperature | Uniform | 102.26 | 87.15 | 6.91 | 0.269 | 34.5 |
| 60° C.-10 d | milky white | | | | | |
| Low temperature | Uniform | 101.77 | 87.26 | 7.64 | 0.236 | 42.8 |
| 4° C.-30 d | milky white | | | | | |
| High temperature | Uniform | 101.34 | 88.16 | 7.12 | 0.243 | 40.6 |
| 40° C.-30 d | milky white | | | | | |
| High temperature | Uniform | 101.97 | 88.04 | 6.58 | 0.246 | 43.5 |
| 60° C.-30 d | milky white | | | | | |
| Low temperature | Uniform | 102.03 | 88.51 | 7.76 | 0.262 | 35.4 |
| cycle for 1 time | milky white | | | | | |
| Low temperature | Uniform | 102.41 | 87.16 | 7.49 | 0.255 | 43.0 |
| cycle for 2 times | milky white | | | | | |
| Low temperature | Uniform | 102.72 | 86.06 | 7.28 | 0.259 | 31.6 |
| cycle for 3 times | milky white | | | | | |

### Example 2: (Strength: 3 mg/mL)

| | | | | | Particle | Zeta |
|---|---|---|---|---|---|---|
| | | Content | Encapsulation | | size | potential |
| | Appearance | % | efficiency % | pH | (μm) | (mV) |
| After sterilization | Uniform | 102.68 | 88.46 | 7.58 | 0.297 | 34.9 |
| | milky white | | | | | |
| Low temperature | Uniform | 102.12 | 88.79 | 7.76 | 0.286 | 40.5 |
| 4° C.-5 d | milky white | | | | | |
| High temperature | Uniform | 102.76 | 85.46 | 7.58 | 0.263 | 38.9 |
| 40° C.-5 d | milky white | | | | | |
| High temperature | Uniform | 103.12 | 86.46 | 7.42 | 0.284 | 41.7 |
| 60° C.-5 d | milky white | | | | | |
| Low temperature | Uniform | 101.16 | 85.15 | 7.73 | 0.301 | 38.1 |
| 4° C.-10 d | milky white | | | | | |
| High temperature | Uniform | 102.24 | 87.49 | 7.53 | 0.298 | 41.7 |
| 40° C.-10 d | milky white | | | | | |
| High temperature | Uniform | 102.02 | 88.52 | 7.28 | 0.300 | 38.0 |
| 60° C.-10 d | milky white | | | | | |
| Low temperature | Uniform | 101.20 | 88.35 | 7.08 | 0.298 | 39.6 |
| 4° C.-30 d | milky white | | | | | |
| High temperature | Uniform | 100.35 | 90.12 | 6.85 | 0.286 | 35.4 |
| 40° C.-30 d | milky white | | | | | |
| High temperature | Uniform | 103.15 | 86.26 | 6.36 | 0.295 | 38.4 |
| 60° C.-30 d | milky white | | | | | |

### Example 3: (Strength: 4 mg/mL)

| | | | | | Particle | Zeta |
|---|---|---|---|---|---|---|
| | | Content | Encapsulation | | size | potential |
| | Appearance | % | efficiency % | pH | (μm) | (mV) |
| After sterilization | Uniform | 100.19 | 87.80 | 7.55 | 0.227 | 32.1 |
| | milky white | | | | | |
| Low temperature | Uniform | 100.36 | 86.30 | 7.68 | 0.232 | 34.9 |
| 4° C.-5 d | milky white | | | | | |
| High temperature | Uniform | 100.37 | 87.25 | 7.42 | 0.222 | 40.7 |
| 40° C.-5 d | milky white | | | | | |
| High temperature | Uniform | 100.77 | 86.93 | 7.33 | 0.234 | 39.1 |
| 60° C.-5 d | milky white | | | | | |
| Low temperature | API | 100.33 | 86.14 | 7.36 | 0.230 | 32.5 |
| 4° C.-10 d | precipitation | | | | | |
| High temperature | Uniform | 100.41 | 87.16 | 7.17 | 0.233 | 36.7 |
| 40° C.-10 d | milky white | | | | | |
| High temperature | Uniform | 101.20 | 88.12 | 6.72 | 0.230 | 39.6 |
| 60° C.-10 d | milky white | | | | | |
| Low temperature | API | 98.87 | 89.27 | 7.59 | 0.236 | 37.0 |
| 4° C.-30 d | precipitation | | | | | |
| High temperature | API | 99.15 | 88.25 | 6.92 | 0.229 | 37.7 |
| 40° C.-30 d | precipitation | | | | | |
| High temperature | API | 99.13 | 87.67 | 6.45 | 0.232 | 37.5 |
| 60° C.-30 d | precipitation | | | | | |
| Low temperature | Uniform | 100.65 | 87.73 | 7.21 | 0.227 | 38.4 |
| cycle for 1 time | milky white | | | | | |
| Low temperature | Uniform | 95.54 | 88.69 | 7.23 | 0.227 | 33.9 |
| cycle for 2 times | milky white | | | | | |
| Low temperature | Uniform | 95.05 | 87.15 | 7.01 | 0.228 | 37.30 |
| cycle for 3 times | milky white | | | | | |

### Example 4: (Strength: 5 mg/mL)

| | | | | | Particle | Zeta |
|---|---|---|---|---|---|---|
| | | Content | Encapsulation | | size | potential |
| | Appearance | % | efficiency % | pH | (μm) | (mV) |
| After sterilization | API | 94.16 | 90.56 | 7.60 | 0.238 | 39.5 |
| | precipitation | | | | | |
| High temperature | API | 91.87 | 89.76 | 7.20 | 0.242 | 38.8 |
| 60° C.-5 d | precipitation | | | | | |
| High temperature | API | 91.16 | 90.18 | 6.98 | 0.24 | 43.5 |
| 60° C.-10 d | precipitation | | | | | |
| High temperature | API | 90.82 | 89.54 | 6.27 | 0.202 | 35.9 |
| 60° C.-30 d | precipitation | | | | | |

Conclusion: after the 2 mg/mL sample was stored for 30 days, the appearance, content, and potential. Indexes such as particle size and the like were all in the qualified range, but the content of the sample of 4 mg/mL was reduced on day 30, and the appearance of the product showed API precipitation (namely active ingredient precipitation) after being stored for 10 days at 4° C. and 20 days at room temperature (see FIG. 4). 0 h after the 5 mg/mL sample was sterilized, the content was low and API precipitation existed.

### 1. Investigation of Different Kinds and Proportions of Oil Phase

Comparative Examples 1-2 were prepared according to the method disclosed in Example 1 of CN94190450.4.

Preparation of oil phase: castor oil and soybean oil or castor oil were/was added to a 250 ml beaker according to the formulation amount and heated in a 70° C. water bath. 2 g of AFSL was weighed out, added to the oil phase, and sheared at 10000 rpm for 2 min, and 12 g of egg yolk lecithin was weighed out, added to the oil phase, and sheared at a speed of 10000 rpm for 5 min to obtain the oil phase.

Preparation of aqueous phase: 861.5 g of water for injection was weighed out, added to a 2000 mL beaker, and warmed in a water bath with the water temperature controlled at 70° C.; 22.5 g of glycerol was added to the aqueous phase and stirred uniformly using a glass rod to obtain the aqueous phase.

Preparation of primary emulsion: the oil phase was added to the aqueous phase while shearing (10000 rpm), and the temperature of the primary emulsion was controlled at 70° C.; after the oil phase was added, the shearing speed was adjusted to 15400 rpm, shearing was performed for 5 min, and the pH of the primary emulsion was adjusted to 9.0 with 0.1 mol/L sodium hydroxide to obtain the primary emulsion.

Preparation of product: low-pressure homogenization pressure: 100 bar/160 bar, homogenization was performed once. High-pressure homogenization pressure: 100 bar/600 bar, homogenization was performed six times. After homogenization, the sample was sealed, filled with nitrogen, and sterilized (121° C., 15 min).

| | Comparative | | Comparative | | |
|---|---|---|---|---|---|
| Formulation | Example 1 | | Example 2 | | |
| Formulation composition | Amount | | | | Use |
| Alphaxalone | 2 | g | 2 | g | Active ingredient |
| Soybean oil (for | / | | 30 | g | Oil phase |
| injection) | | | | | |
| Castor oil | 100 | g | 70 | g | Oil phase |
| Egg yolk lecithin | 12 | g | 12 | g | Emulsifier |
| Glycerol | 22.5 | g | 22.5 | g | Osmotic pressure |
| | | | | | regulator |
| Water for injection | 861.5 | g | 861.5 | g | Aqueous phase |

### Experimental Results

| | Comparative | Comparative |
|---|---|---|
| Item | Example 1 | Example 2 |
| Content | 100.56 | 101.29 |
| Encapsulation efficiency | 91.2 | 89.03 |
| pH | 7.54 | 7.45 |
| Particle size (μm) | 0.548 | 0.459 |
| Zeta potential (mV) | 41.4 | 39.6 |

Conclusion: according to the experimental process, 10% castor oil can completely dissolve AFSL, and indexes such as content, potential and the like were in a qualified range, but the particle size of the emulsion sample was more than 0.5 μm, which was in an unqualified range according to General Chapter 0102, Chinese Pharmacopoeia, Volume IV.

The feasibility of preparing samples of different formulation compositions was attempted with reference to the test method of Example 1.

Formulation composition (Examples 5-9, preparation volume 1000 mL)

| | | | | | | | | | | | | | | | Comparative | | |
|---|---|---|---|---|---|---|---|---|---|---|---|---|---|---|---|---|---|
| | | | | | | | | | | | | | | | Example 3, | | |
| | | | Example 6 | | | | Example 8 | | Example 9 | | | | | | prepared | | |
| | | | 10% oil | | Example | | 20% oil | | 30% oil | | Comparative | | Comparative | | according to | | |
| Formulation | Example 5 | | phase | | 7 | | phase | | phase | | Example 1 | | Example 2 | | the method of | | |
| Formulation | 10% oil | | | | | | | | | | | | | | Comparative | | |
| composition | phase | | Amount | | | | | | | | | | | | Example 2 | | Use |
| Alphaxalone | 2 | g | 2 | g | 2 | g | 2 | g | 2 | g | 2 | g | 2 | g | 2 | g | Active |
| | | | | | | | | | | | | | | | | | ingredient |
| Soybean oil | 100 | g | 50 | g | 100 | g | 200 | g | 300 | g | / | | 30 | g | 60 | g | Oil phase |
| (for injection) | | | | | | | | | | | | | | | | | |
| Medium-chain | 0 | g | 50 | g | 100 | g | 0 | g | 0 | g | / | | / | | / | | Oil phase |
| triglyceride | | | | | | | | | | | | | | | | | |
| Castor oil | / | | / | | / | | / | | / | | 100 | g | 70 | g | 140 | g | |
| Egg yolk | 12 | g | 12 | g | 12 | g | 12 | g | 12 | g | 12 | g | 12 | g | 12 | g | Emulsifier |
| lecithin | | | | | | | | | | | | | | | | | |
| Glycerol | 25 | g | 25 | g | 25 | g | 25 | g | 25 | g | 22.5 | g | 22.5 | g | 22.5 | g | Osmotic |
| | | | | | | | | | | | | | | | | | pressure |
| | | | | | | | | | | | | | | | | | regulator |

| | | | | | | | | | | | | | | |
|---|---|---|---|---|---|---|---|---|---|---|---|---|---|---|
| Sodium oleate | 0.3 | g | 0.3 | g | 0.3 | g | 0.3 | g | 0.3 | g | / | / | / | Stabilizer |

| | | | | | | | | | |
|---|---|---|---|---|---|---|---|---|---|
| Sodium | Proper | Proper | Proper | Proper | Proper | Proper | Proper | Proper | pH adjuster |
| hydroxide | amount | amount | amount | amount | amount | amount | amount | amount | |
| Water for | Proper | Proper | Proper | Proper | Proper | Proper | Proper | Proper | Aqueous |
| injection | amount | amount | amount | amount | amount | amount | amount | amount | phase |

### Experimental Results

| | | | | | Particle | Zeta |
|---|---|---|---|---|---|---|
| | | Content | Encapsulation | | size | potential |
| Example No. | Appearance | % | efficiency % | pH | (μm) | (mV) |
| Example 5 | Uniform milky white | 99.76 | 88.25 | 7.76 | 0.185 | 38.5 |
| Example 6 | Uniform milky white | 99.89 | 87.92 | 7.79 | 0.176 | 39.2 |
| Example 7 | Uniform milky white | 102.35 | 85.76 | 7.70 | 0.262 | 33.3 |
| Example 8 | Uniform milky white | 99.20 | 86.78 | 8.00 | 0.321 | 38.7 |
| Example 9 | Uniform milky white | 100.89 | 87.54 | 7.94 | 0.409 | 46.5 |
| Comparative | Uniform milky white | 100.56 | 91.23 | 7.54 | 0.548 | 41.4 |
| Example 1 | | | | | | |
| Comparative | Uniform milky white | 101.29 | 89.03 | 7.45 | 0.459 | 39.6 |
| Example 2 | | | | | | |
| Comparative | Uniform milky white | 99.78 | 93.36 | 7.36 | 0.470 | 35.6 |
| Example 3 | | | | | | |

Conclusion: the sample prepared by using different oil phase (soybean oil or a mixture of soybean oil and medium-chain triglyceride) proportions (10%, 20%, and 30%) had no significant difference in properties, content, encapsulation efficiency, pH, and Zeta potential, but the average particle sizes of the samples were gradually increased with the increase of the oil phase proportion, wherein the average particle size of the formulation containing 30% oil phase was larger than 0.4 μm, which was close to the limit. The samples containing the castor oil formulation had the indexes of properties, encapsulation efficiency, pH, content, potential, and the like within qualified ranges, but had average particle sizes larger than or close to 0.5 μm, which were significantly higher than those of the castor oil-free formulation.

In conclusion, the selection of the type and the proportion of the oil for injection should be within a proper range to ensure that the emulsifier can completely emulsify the oil phase and the aqueous phase. Therefore, in the present disclosure, the oil phase preferably accounts for 10-20%; preferably, the oil phase is soybean oil or a mixture of soybean oil and medium-chain triglyceride. The following examples were investigated with an oil phase ratio of 20% (the ratio of soybean oil to medium-chain triglyceride was 1:1).

### 2. Investigation of Different Phospholipid Ratios

2.1.1 Formulations with different proportions of phospholipids were attempted at 2 mg/mL to investigate the feasibility of the samples. The preparation method was according to Example 1 (batch size 1000 mL)

| Formulation | Example 10 | | Example 11 | | Example 12 | | Example 13 | | |
|---|---|---|---|---|---|---|---|---|---|
| composition | 0.6% phospholipid | | 0.9% phospholipid | | 1.2% phospholipid | | 1.5% phospholipid | | Use |
| Alphaxalone | 2 | g | 2 | g | 2 | g | 2 | g | Active |
| | | | | | | | | | ingredient |
| Soybean oil (for | 100 | g | 100 | g | 100 | g | 100 | g | Oil phase |
| injection) | | | | | | | | | |
| Medium-chain | 100 | g | 100 | g | 100 | g | 100 | g | Oil phase |
| triglyceride | | | | | | | | | |
| Egg yolk lecithin | 6 | g | 9 | g | 12 | g | 15 | g | Emulsifier |
| Glycerol | 25 | g | 25 | g | 25 | g | 25 | g | Osmotic |
| | | | | | | | | | pressure |
| | | | | | | | | | regulator |
| Sodium oleate | 0.3 | g | 0.3 | g | 0.3 | g | 0.3 | g | Stabilizer |

| | | | | | |
|---|---|---|---|---|---|
| Sodium hydroxide | Proper amount | Proper amount | Proper amount | Proper amount | pH adjuster |
| Water for injection | Proper amount | Proper amount | Proper amount | Proper amount | Aqueous |
| | | | | | phase |

### 2.1.2 Sample Detection Results:

| | | Example 11 | Example 12 | Example 13 |
|---|---|---|---|---|
| | Example 10 | Uniform | Uniform | Uniform |
| Item | Oil- | milky | milky | milky |
| Appearance | floating | white | white | white |
| Content | 102.56 | 100.53 | 102.35 | 99.92 |
| Encapsulation | 88.00 | 87.71 | 85.76 | 86.57 |
| efficiency | | | | |
| pH | 7.40 | 7.75 | 7.70 | 7.62 |
| Particle | 0.360 | 0.261 | 0.262 | 0.248 |
| size (μm) | | | | |
| Zeta potential | 40.2 | 38.6 | 33.3 | 38.2 |
| (mV) | | | | |

2.1.3 Conclusion: in the range of 0.6%-1.5% of phospholipid, the content and the potential were not significantly different, and the particle size of a sample tended to be reduced with the increase of the phospholipid proportion, but the difference was not large in the range of 0.9%-1.5%; the sample containing 0.6% phospholipid showed slight oil floating.

### 2.1.4 Results of Influencing Factors:

### Example 10 Sample Batch

| | | | Particle | Zeta |
|---|---|---|---|---|
| | Content | | size | potential |
| | (%) | pH | (μm) | (mV) |
| After sterilization | 102.56 | 7.40 | 0.360 | 40.2 |
| Low temperature 4° C.-5 d | 101.80 | 7.25 | 0.409 | 38.1 |
| High temperature 40° C.-5 d | 101.34 | 7.24 | 0.420 | 38.4 |
| High temperature 60° C.-5 d | 101.50 | 7.11 | 0.416 | 38.3 |
| Low temperature 4° C.-10 d | 101.36 | 7.16 | 0.407 | 38.1 |
| High temperature 40° C.-10 d | 101.66 | 7.13 | 0.418 | 38.5 |
| High temperature 60° C.-10 d | 102.45 | 7.00 | 0.415 | 39.1 |
| Low temperature 4° C.-30 d | 100.25 | 6.85 | 0.420 | 39.6 |
| High temperature 40° C.-30 d | 100.26 | 6.64 | 0.423 | 38.6 |
| High temperature 60° C.-30 d | 101.25 | 6.38 | 0.422 | 39.5 |

### Example 11 Sample Batch

| | | | Particle | Zeta |
|---|---|---|---|---|
| | Content | | size | potential |
| | (%) | pH | (μm) | (mV) |
| After sterilization | 100.53 | 7.75 | 0.261 | 38.6 |
| Low temperature 4° C.-5 d | 100.41 | 7.66 | 0.269 | 40.9 |
| High temperature 40° C.-5 d | 101.48 | 7.44 | 0.263 | 36.4 |
| High temperature 60° C.-5 d | 101.47 | 7.25 | 0.258 | 40.6 |
| Low temperature 4° C.-10 d | 101.71 | 7.40 | 0.265 | 39.6 |
| High temperature 40° C.-10 d | 100.42 | 7.26 | 0.265 | 40.3 |
| High temperature 60° C.-10 d | 101.23 | 7.09 | 0.258 | 40.6 |
| Low temperature 4° C.-30 d | 101.26 | 6.86 | 0.265 | 39.6 |
| High temperature 40° C.-30 d | 102.02 | 6.75 | 0.261 | 38.5 |
| High temperature 60° C.-30 d | 100.02 | 6.60 | 0.262 | 40.2 |

### Example 12 Sample Batch

| | | | Particle | Zeta |
|---|---|---|---|---|
| | Content | | size | potential |
| | (%) | pH | (μm) | (mV) |
| After sterilization | 102.35 | 7.70 | 0.262 | 33.3 |
| Low temperature 4° C.-5 d | 101.37 | 7.75 | 0.252 | 38.5 |
| High temperature 40° C.-5 d | 102.19 | 7.65 | 0.252 | 39.8 |
| High temperature 60° C.-5 d | 102.78 | 7.34 | 0.255 | 41.2 |
| Low temperature 4° C.-10 d | 102.73 | 7.70 | 0.267 | 33.9 |
| High temperature 40° C.-10 d | 101.86 | 7.27 | 0.256 | 37.1 |
| High temperature 60° C.-10 d | 102.26 | 6.91 | 0.269 | 34.5 |
| Low temperature 4° C.-30 d | 101.77 | 7.64 | 0.236 | 42.8 |
| High temperature 40° C.-30 d | 101.34 | 7.12 | 0.243 | 40.6 |
| High temperature 60° C.-30 d | 101.97 | 6.58 | 0.246 | 43.5 |

### Example 13 Sample Batch

| | | | Particle | Zeta |
|---|---|---|---|---|
| | Content | | size | potential |
| | (%) | pH | (μm) | (mV) |
| After sterilization | 99.92 | 7.62 | 0.248 | 38.2 |
| Low temperature 4° C.-5 d | 99.93 | 7.62 | 0.244 | 35.2 |
| High temperature 40° C.-5 d | 99.76 | 7.53 | 0.244 | 35.2 |
| High temperature 60° C.-5 d | 100.86 | 7.21 | 0.246 | 34.4 |
| Low temperature 4° C.-10 d | 101.71 | 7.70 | 0.248 | 38.3 |
| High temperature 40° C.-10 d | 100.48 | 7.37 | 0.248 | 36.6 |
| High temperature 60° C.-10 d | 101.60 | 7.18 | 0.256 | 37.4 |
| Low temperature 4° C.-30 d | 101.02 | 6.85 | 0.245 | 39.2 |
| High temperature 40° C.-30 d | 99.98 | 6.67 | 0.249 | 37.5 |
| High temperature 60° C.-30 d | 101.52 | 6.40 | 0.251 | 39.5 |

2.1.5 Conclusion: compared with day 0, the samples containing 0.9%-1.5% of phospholipid had stable results in 30 days of each influencing factor, and indexes such as content, particle size, potential, and the like had no significant change. However, in the sample containing 0.6% phospholipid, the influence factor results showed that the average particle size of the sample increased and aggregation tended to occur. It showed that the emulsion sample was unstable.

### 3. Investigation of Different Emulsifiers

3.1 Because emulsion samples of 4 mg/mL and 5 mg/mL strengths prepared without adding a sodium glycocholate formulation showed API precipitation during the stability sample storage, different coemulsifiers (poloxamer 188, sodium glycocholate, Tween 80, or 15-hydroxystearic acid polyethylene glycol ester (HS15)) and phospholipid were added as composite emulsifiers in an attempt to investigate the feasibility of the emulsion sample of 5 mg/mL.

### Example 16: Preparation Method of Emulsions with Sodium Glycocholate (Batch Size 1000 mL)

### Preparation of Oil Phase:

Soybean oil and medium-chain triglyceride were each weighed out according to the formulation amount and heated in a 60° C. water bath. Egg yolk lecithin was weighed out according to the formulation amount and added to the oil phase, and AFSL was weighed out, added to the oil phase, and sheared at 10000 rpm for 5 min.

### Preparation of Aqueous Phase:

A proper amount of water for injection was weighed out and added to a 2000 mL beaker, and warmed in a water bath with the water temperature controlled at 60° C., and sodium glycocholate, sodium oleate, and glycerol were weighed out according to the formulation amount, added to the aqueous phase, and stirred uniformly to obtain the aqueous phase.

### Preparation of Primary Emulsion:

The oil phase was added to the aqueous phase while shearing (10000 rpm), and the temperature of the primary emulsion was controlled at 60° C.; after the oil phase was added, the shearing speed was adjusted to 15400 rpm, and shearing was performed for 5 min. The pH of the primary emulsion was adjusted to 9 with 0.5 mol/L sodium hydroxide.

### Preparation of Product:

Low-pressure homogenization pressure: 100 bar/160 bar, homogenization was performed once. High-pressure homogenization pressure: 100 bar/600 bar, homogenization was performed six times. After homogenization, the sample was sealed, filled with nitrogen, and sterilized (121° C., 15 min).

Examples 14, 15, and 17 were prepared according to the method of Example 16, replacing only the coemulsifier.

| Formulation | Example 14 | Example 15 | Example 16 | Example 17 | |
|---|---|---|---|---|---|
| Formulation composition | Amount | | | | Use |
| Alphaxalone | 5 g | 5 g | 5 g | 5 g | Active |
| | | | | | ingredient |
| Soybean oil (for | 100 g | 100 g | 100 g | 100 g | Oil phase |
| injection) | | | | | |
| Medium-chain | 100 g | 100 g | 100 g | 100 g | Oil phase |
| triglyceride | | | | | |
| Egg yolk | 12 g | 12 g | 12 g | 12 g | Emulsifier |
| lecithin | | | | | |
| Poloxamer 188 | 6 g | / | / | / | Coemulsifier |
| HS15 | / | 6 g | / | | Coemulsifier |
| Tween 80 | / | / | / | 6 g | Coemulsifier |
| Sodium | / | / | 6 g | / | Coemulsifier |
| glycocholate | | | | | |
| Glycerol | 25 g | 25 g | 25 g | 25 g | Osmotic |
| | | | | | pressure |
| | | | | | regulator |
| Sodium oleate | 0.3 g | 0.3 g | 0.3 g | 0.3 g | Stabilizer |
| Sodium | Proper | Proper | Proper | Proper | pH adjuster |
| hydroxide | amount | amount | amount | amount | |
| Water for | Proper | Proper | Proper | Proper | Aqueous |
| injection | amount | amount | amount | amount | phase |

### 3.2. Experimental Results

| | | | | | Particle | Zeta |
|---|---|---|---|---|---|---|
| | | Content | Encapsulation | | size | potential |
| | Appearance | % | efficiency % | pH | (μm) | (mV) |
| Example 14 | Oil-floating | 102.93 | 90.47 | 7.30 | 0.152 | 37.9 |
| Example 15 | API precipitation | 99.83 | / | 7.5 | 0.160 | 39.7 |
| Example 16 | Milky white | 100.51 | 90.94 | 7.82 | 0.146 | 36.6 |
| Example 17 | API precipitation | 99.51 | / | 7.42 | 0.196 | 38.6 |

3.3 Conclusion: compared with the formulation in Example 16, the formulation in Example 14 had oil-floating appearance, and the formulation in Example 15 and Example 17 had API precipitation appearance, which shows that Tween 80, poloxamer 188, and HS15 can not be used as a coemulsifier. The appearance of the formulation in Example 16 was in accordance with the specification, and the indexes of content, potential, particle size, and the like were in the qualified range. In conclusion, formulations containing sodium glycocholate can improve the stability of an emulsion sample.

### 3.4 Formulation Composition (Strength: 4 mg/mL)

| Formulation composition | Example 18 | Example 19 | Example 20 | Use |
|---|---|---|---|---|
| Alphaxalone | 4 g | 4 g | 4 g | Active ingredient |
| Soybean oil (for | 100 g | 100 g | 100 g | Oil phase |
| injection) | | | | |
| Medium-chain | 100 g | 100 g | 100 g | Oil phase |
| triglyceride | | | | |
| Egg yolk lecithin | 12 g | 12 g | 12 g | Emulsifier |
| Glycerol | 25 g | 25 g | 25 g | Osmotic pressure |
| | | | | regulator |
| Poloxamer 188 | 6 g | / | / | Coemulsifier |
| HS15 | / | 6 g | / | Coemulsifier |
| Tween 80 | / | / | 6 g | Coemulsifier |
| Sodium hydroxide | Proper | Proper | Proper | pH adjuster |
| | amount | amount | amount | |
| Sodium oleate | 0.3 g | 0.3 g | 0.3 g | Stabilizer |
| Water for injection | Proper | Proper | Proper | Aqueous phase |
| | amount | amount | amount | |

| Formulation composition | Example 21 | Example 22 | Example 23 | Use |
|---|---|---|---|---|
| Alphaxalone | 4 g | 4 g | 4 g | Active ingredient |
| Soybean oil (for | 100 g | 100 g | 100 g | Oil phase |
| injection) | | | | |
| Medium-chain | 100 g | 100 g | 100 g | Oil phase |
| triglyceride | | | | |
| Egg yolk lecithin | 12 g | 12 g | 12 g | Emulsifier |
| Glycerol | 25 g | 25 g | 25 g | Osmotic pressure |
| | | | | regulator |
| Glycocholic acid | 6 g | / | / | Coemulsifier |
| Cholic acid | / | 6 g | / | Coemulsifier |
| Sodium taurocholate | / | / | 6 g | Coemulsifier |
| Sodium hydroxide | Proper | Proper | Proper | pH adjuster |
| | amount | amount | amount | |
| Sodium oleate | 0.3 g | 0.3 g | 0.3 g | Stabilizer |
| Water for injection | Proper | Proper | Proper | Aqueous phase |
| | amount | amount | amount | |

### 3.5 Detection Results of Samples

| | | | | Particle | Zeta |
|---|---|---|---|---|---|
| | | Content | | size | potential |
| Item | Appearance | (%) | pH | (μm) | (mV) |
| Example 18 | Oil-floating | 102.93 | 7.30 | 0.152 | 37.9 |
| Example 19 | API precipitation | 99.83 | 7.50 | 0.160 | 39.7 |
| Example 20 | API precipitation | 99.51 | 7.42 | 0.196 | 38.6 |
| Example 21 | Uniform | 100.51 | 7.52 | 0.269 | 36.6 |
| | milky white | | | | |
| Example 22 | Uniform | 100.36 | 7.31 | 0.272 | 57.1 |
| | milky white | | | | |
| Example 23 | Uniform | 102.11 | 7.58 | 0.234 | 57.4 |
| | milky white | | | | |

3.6 Conclusion: the sample in Example 18 had oil-floating appearance, and the formulation in Example 19 and Example 20 had API precipitation appearance, which shows that Tween 80, poloxamer 188, and HS15 can not be used as a cosolvent. The appearance of the sample in Examples 21-23 was in accordance with the specification, and the indexes of content, potential, particle size, and the like were in the qualified range. In conclusion, the formulation containing a cholic acid compound or a salt thereof can enhance the stability of emulsion samples.

### 4. Confirmation of Effect of Sodium Glycocholate

### 4.1 Comparison Between without Sodium Glycocholate and with Sodium Glycocholate (Batch Size 1000 mL)

| | Example 24 | Example 25 | |
|---|---|---|---|
| | (Without | (0.4% | |
| Formulation | sodium | sodium | |
| composition | glycocholate) | glycocholate) | Use |
| Alphaxalone | 4 g | 4 g | Active |
| | | | ingredient |
| Soybean oil (for | 100 g | 100 g | Oil phase |
| injection) | | | |
| Medium-chain | 100 g | 100 g | Oil phase |
| triglyceride | | | |
| Egg yolk lecithin | 12 g | 12 g | Emulsifier |
| Sodium | 0 g | 4 g | Coemulsifier |
| glycocholate | | | |
| Glycerol | 25 g | 25 g | Osmotic |
| | | | pressure |
| | | | regulator |
| Sodium oleate | 0.3 g | 0.3 g | Stabilizer |
| Sodium | Proper | Proper | pH adjuster |
| hydroxide | amount | amount | |
| Water for | Proper | Proper | Aqueous |
| injection | amount | amount | phase |

### 4.2 Test Results of Samples

| | Example 24 | Example 25 |
|---|---|---|
| | (Without | (0.4% |
| | sodium | sodium |
| Item | glycocholate) | glycocholate) |
| Content | 100.19 | 103.77 |
| Encapsulation efficiency | 87.8 | 91.72 |
| pH | 7.55 | 7.85 |
| Particle size (μm) | 0.227 | 0.154 |
| Zeta potential (mV) | 32.1 | 39.1 |

4.3 Conclusion: the indexes such as content, potential, encapsulation efficiency, and the like of two formulation samples were not significantly different, and the average particle size of the sample added with the sodium glycocholate formulation was smaller than that of a conventional formulation.

### 4.4 Stability Data

### Example 24 Sample Batch

| | | | | Particle | Zeta |
|---|---|---|---|---|---|
| | | Content | | size | potential |
| | Appearance | (%) | pH | (μm) | (mV) |
| After sterilization | Uniform | 100.19 | 7.55 | 0.227 | 32.1 |
| | milky white | | | | |
| Low temperature | Uniform | 100.36 | 7.68 | 0.232 | 34.9 |
| 4° C.-5 d | milky white | | | | |
| High temperature | Uniform | 100.37 | 7.42 | 0.222 | 40.7 |
| 40° C.-5 d | milky white | | | | |
| High temperature | Uniform | 100.77 | 7.33 | 0.234 | 39.1 |
| 60° C.-5 d | milky white | | | | |
| Low temperature | API | 100.33 | 7.36 | 0.230 | 32.5 |
| 4° C.-10 d | precipitation | | | | |
| High temperature | Uniform | 100.41 | 7.17 | 0.233 | 36.7 |
| 40° C.-10 d | milky white | | | | |
| High temperature | Uniform | 101.20 | 6.72 | 0.230 | 39.6 |
| 60° C.-10 d | milky white | | | | |
| Low temperature | API | 98.87 | 7.59 | 0.236 | 37.0 |
| 4° C.-30 d | precipitation | | | | |
| High temperature | API | 99.15 | 6.92 | 0.229 | 37.7 |
| 40° C.-30 d | precipitation | | | | |
| High temperature | API | 99.13 | 6.45 | 0.232 | 37.5 |
| 60° C.-30 d | precipitation | | | | |
| Low temperature | Uniform | 100.65 | 7.21 | 0.227 | 38.4 |
| cycle for 1 time | milky white | | | | |
| Low temperature | Uniform | 95.54 | 7.23 | 0.227 | 33.9 |
| cycle for 2 times | milky white | | | | |
| Low temperature | Uniform | 95.05 | 7.01 | 0.228 | 37.30 |
| cycle for 3 times | milky white | | | | |

### Example 25 Sample Batch

| | | | | Particle | Zeta |
|---|---|---|---|---|---|
| | | Content | | size | potential |
| | Appearance | (%) | pH | (μm) | (mV) |
| After sterilization | Uniform milky white | 103.77 | 7.85 | 0.154 | 39.1 |
| Low temperature 4° C.- | Uniform milky white | 103.45 | 7.67 | 0.153 | 43.2 |
| 5 d | | | | | |
| High temperature | Uniform milky white | 103.24 | 7.62 | 0.153 | 46.1 |
| 40° C.-5 d | | | | | |
| High temperature | Uniform milky white | 103.12 | 7.48 | 0.154 | 46.3 |
| 60° C.-5 d | | | | | |
| Low temperature 4° C.- | Uniform milky white | 103.62 | 7.31 | 0.154 | 42.9 |
| 10 d | | | | | |
| High temperature | Uniform milky white | 103.41 | 7.29 | 0.153 | 42.9 |
| 40° C.-10 d | | | | | |
| High temperature | Uniform milky white | 103.24 | 7.08 | 0.153 | 39.6 |
| 60° C.-10 d | | | | | |
| Low temperature 4° C.- | Uniform milky white | 104.33 | 7.81 | 0.157 | 44.1 |
| 30 d | | | | | |
| High temperature | Uniform milky white | 102.67 | 7.51 | 0.157 | 45.9 |
| 40° C.-30 d | | | | | |
| High temperature | Uniform milky white | 100.28 | 7.01 | 0.154 | 43.0 |
| 60° C.-30 d | | | | | |
| Low temperature cycle | Uniform milky white | 103.92 | 7.65 | 0.153 | 39.5 |
| for 1 time | | | | | |
| Low temperature cycle | Uniform milky white | 102.68 | 7.72 | 0.154 | 40.1 |
| for 2 times | | | | | |
| Low temperature cycle | Uniform milky white | 101.50 | 7.53 | 0.152 | 40.2 |
| for 3 times | | | | | |

4.5 Conclusion: from the stability data, the indexes such as content, potential, average particle size, and the like of the sodium glycocholate-containing formulation had no significant change when the sample was stored for 30 days compared with 0. The content of the sample without adding the sodium glycocholate formulation was low at low temperature on day 10, and the appearance of the sample had an API precipitation phenomenon. This indicates that the addition of sodium glycocholate contributes to the stability of the emulsion samples.

5. Investigation of Feasibility of Preparing Formulations with Larger Strengths (Containing Glycocholic Acid)

### Preparation Method

### Preparation of Oil Phase:

Soybean oil and medium-chain triglyceride were each weighed out according to the formulation amount and heated in a 60° C. water bath. Egg yolk lecithin was weighed out according to the formulation amount and added to the oil phase, and AFSL was weighed out, added to the oil phase, and sheared at 10000 rpm for 5 min.

### Preparation of Aqueous Phase:

Water for injection was weighed out according to the formulation amount and added to a 2000 mL beaker, and warmed in a water bath with the water temperature controlled at 60° C., glycocholic acid was weighed out according to the formulation amount and added to the water for injection, the aqueous phase was adjusted to be clear with 0.5 mol/L sodium hydroxide, and sodium oleate and glycerol were weighed out according to the formulation amount, added to the aqueous phase, and stirred until uniformly mixed to obtain the aqueous phase.

### Preparation of Primary Emulsion:

The oil phase was added to the aqueous phase while shearing (10000 rpm), and the temperature of the primary emulsion was controlled at 60° C.; after the oil phase was added, the shearing speed was adjusted to 15400 rpm, and shearing was performed for 5 min. The pH of the primary emulsion was adjusted to 9 with 0.5 mol/L sodium hydroxide.

### Preparation of Product:

Low-pressure homogenization pressure: 100 bar/160 bar, homogenization was performed once. High-pressure homogenization pressure: 100 bar/600 bar, homogenization was performed six times. After homogenization, the sample was sealed, filled with nitrogen, and sterilized (121° C., 15 min).

### 5.1 Batch Size 1000 mL

| | Example 26 | Example 27 | |
|---|---|---|---|
| | (5 mg/mL) | (6 mg/mL) | |
| | 0.4% | 0.4% | |
| Formulation | Glycocholic | Glycocholic | |
| composition | acid | acid | Use |
| Alphaxalone | 5 g | 6 g | Active |
| | | | ingredient |
| Soybean oil (for | 100 g | 100 g | Oil phase |
| injection) | | | |
| Medium-chain | 100 g | 100 g | Oil phase |
| triglyceride | | | |
| Egg yolk lecithin | 12 g | 12 g | Emulsifier |
| Sodium oleate | 0.3 g | 0.3 g | Stabilizer |
| Glycocholic acid | 4 g | 4 g | Coemulsifier |
| Glycerol | 25 g | 25 g | Osmotic |
| | | | pressure |
| | | | regulator |
| Sodium | Proper | Proper | pH adjuster |
| hydroxide | amount | amount | |
| Water for | Proper | Proper | Aqueous |
| injection | amount | amount | phase |

### 5.2 Detection Results of Samples

| | Example 26 | Example 27 |
|---|---|---|
| | (5 mg/mL) | (6 mg/mL) |
| | 0.4% | 0.4% |
| Item | Glycocholic acid | Glycocholic acid |
| Content | 100.45 | 98.13 |
| Encapsulation efficiency | 90.88 | 91.12 |
| pH | 7.80 | 7.70 |
| Particle size (μm) | 0.154 | 0.155 |
| Zeta potential (mV) | 45.3 | 39.9 |

5.3 Conclusion: the indexes of sample content, appearance, potential, particle size, and the like of 5 mg/mL and 6 mg/mL met the requirements.

### 6. Investigation of Amount of Sodium Glycocholate

### 6.1 Investigation of the Influence of Different Proportions of Sodium Glycocholate on the Stability of the Samples in the Case of 4 mg/mL Strength (Batch Size 1000 mL)

| | Example 28 | Example 29 | Example 30 | Example 31 | Example 32 |
|---|---|---|---|---|---|
| Formulation | 0.05% sodium | 0.1% sodium | 0.14% sodium | 0.2% sodium | 0.4% sodium |
| composition | glycocholate | glycocholate | glycocholate | glycocholate | glycocholate |
| Alphaxalone | 4 g | 4 g | 4 g | 4 g | 4 g |
| Soybean oil (for | 100 g | 100 g | 100 g | 100 g | 100 g |
| injection) | | | | | |
| Medium-chain | 100 g | 100 g | 100 g | 100 g | 100 g |
| triglyceride | | | | | |
| Egg yolk | 12 g | 12 g | 12 g | 12 g | 12 g |
| lecithin | | | | | |
| Sodium | 0.5 g | 1 g | 1.4 g | 2 g | 4 g |
| glycocholate | | | | | |
| Sodium oleate | 0.3 g | 0.3 g | 0.3 g | 0.3 g | 0.3 g |
| Glycerol | 25 g | 25 g | 25 g | 25 g | 25 g |
| Water for | Proper | Proper | Proper | Proper | Proper |
| injection | amount | amount | amount | amount | amount |

| | Example 33 | Example 34 | Example 35 | Example 36 | Example 37 |
|---|---|---|---|---|---|
| Formulation | 0.8% sodium | 1.0% sodium | 1.2% sodium | 1.4% sodium | 2.0% sodium |
| composition | glycocholate | glycocholate | glycocholate | glycocholate | glycocholate |
| Alphaxalone | 4 g | 4 g | 4 g | 4 g | 4 g |
| Soybean oil (for | 100 g | 100 g | 100 g | 100 g | 100 g |
| injection) | | | | | |
| Medium-chain | 100 g | 100 g | 100 g | 100 g | 100 g |
| triglyceride | | | | | |
| Egg yolk | 12 g | 12 g | 12 g | 12 g | 12 g |
| lecithin | | | | | |
| Sodium | 8 g | 10 g | 12 g | 14 g | 20 g |
| glycocholate | | | | | |
| Sodium oleate | 0.3 g | 0.3 g | 0.3 g | 0.3 g | 0.3 g |
| Glycerol | 25 g | 25 g | 25 g | 25 g | 25 g |
| Water for | Proper | Proper | Proper | Proper | Proper |
| injection | amount | amount | amount | amount | amount |

### 6.2 Detection Results of Samples

| | Appearance | | | | Particle | Zeta |
|---|---|---|---|---|---|---|
| | of primary | Appearance | Content | | size | potential |
| | emulsion | of product | (%) | pH | (μm) | (mV) |
| Example 28 | Uniform | Uniform | 102.95 | 7.96 | 0.216 | 39.8 |
| 0.05% | milky white | milky white | | | | |
| Example 29 | Uniform | Uniform | 100.30 | 7.62 | 0.210 | 41.3 |
| 0.1% | milky white | milky white | | | | |
| Example 30 | Uniform | Uniform | 101.20 | 7.55 | 0.202 | 42.1 |
| 0.14% | milky white | milky white | | | | |
| Example 31 | Uniform | Uniform | 99.60 | 7.69 | 0.208 | 41.1 |
| 0.2% | milky white | milky white | | | | |
| Example 32 | Uniform | Uniform | 103.77 | 7.85 | 0.154 | 39.1 |
| 0.4% | milky white | milky white | | | | |
| Example 33 | Uniform | Uniform | 99.86 | 7.55 | 0.154 | 48.7 |
| 0.8% | milky white | milky white | | | | |
| Example 34 | Uniform | Uniform | 101.31 | 7.58 | 0.155 | 40.2 |
| 1.0% | milky white | milky white | | | | |
| Example 35 | Creaming | Uniform | 100.10 | 7.70 | 0.154 | 41.2 |
| 1.2% | | milky white | | | | |
| Example 36 | Creaming | Uniform | 101.24 | 7.72 | 0.156 | 40.7 |
| 1.4% | | milky white | | | | |
| Example 37 | Creaming | Uniform | 100.12 | 7.30 | 0.152 | 39.0 |
| 2.0% | | milky white | | | | |

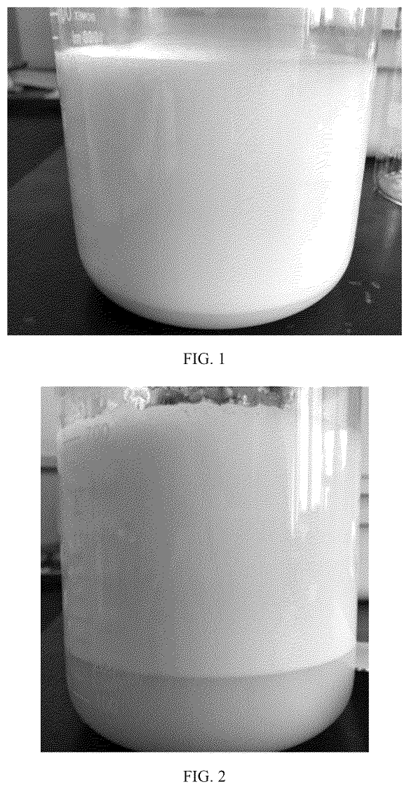

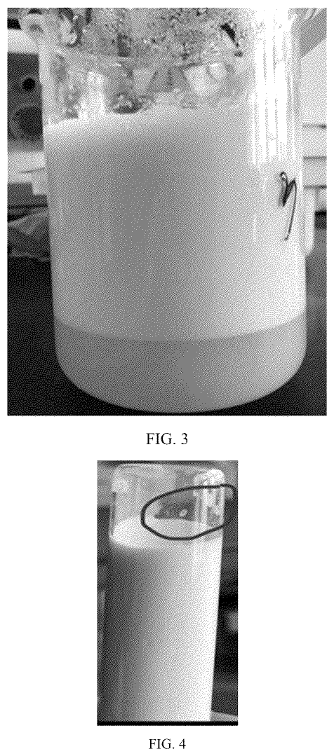

6.3 Conclusion: with the increase of the content of the sodium glycocholate, the indexes such as content, potential, and the like of the emulsion samples were not significantly different, but the average particle size of the samples tended to be gradually reduced. When the content of the sodium glycocholate in each milliliter of the alphaxalone fat emulsion injection was, for example, 12 mg, 14 mg, or 20 mg, creaming was found in the primary emulsion (see FIGS. 1, 2, and 3), and when the content of the sodium glycocholate in each milliliter of the alphaxalone fat emulsion injection was 0.5-10 mg, the appearance and other characteristics of the sample primary emulsion were relatively good. Stability experiments were performed on emulsion samples with sodium glycocholate content of 0.5-10 mg in each milliliter of the alphaxalone fat emulsion injection.

### 6.4 Stability Data

### Example 28 Sample Batch (0.05%)

| | | | | Particle | Zeta |
|---|---|---|---|---|---|
| | | Content | | size | potential |
| | Appearance | (%) | pH | (μm) | (mV) |
| After | Uniform milky | 102.95 | 7.96 | 0.216 | 39.8 |
| sterilization | white | | | | |
| Low temperature | Uniform milky | 102.45 | 7.82 | 0.215 | 40.3 |
| 4° C.-5 d | white | | | | |
| High temperature | Uniform milky | 101.24 | 7.76 | 0.216 | 45.1 |
| 40° C.-5 d | white | | | | |
| High temperature | Uniform milky | 101.12 | 7.58 | 0.217 | 43.3 |
| 60° C.-5 d | white | | | | |
| Low temperature | Uniform milky | 101.62 | 7.61 | 0.214 | 40.9 |
| 4° C.-10 d | white | | | | |
| High temperature | Uniform milky | 101.41 | 7.49 | 0.215 | 41.9 |
| 40° C.-10 d | white | | | | |
| High temperature | Uniform milky | 101.24 | 7.38 | 0.215 | 39.8 |
| 60° C.-10 d | white | | | | |
| Low temperature | Uniform milky | 102.33 | 7.41 | 0.216 | 40.1 |
| 4° C.-30 d | white | | | | |
| High temperature | Uniform milky | 102.67 | 7.31 | 0.215 | 42.9 |
| 40° C.-30 d | white | | | | |
| High temperature | Uniform milky | 100.28 | 7.01 | 0.215 | 41.0 |
| 60° C.-30 d | white | | | | |

### Example 29 Sample Batch (0.1%)

| | | | | Particle | Zeta |
|---|---|---|---|---|---|
| | | Content | | size | potential |
| | Appearance | (%) | pH | (μm) | (mV) |
| After | Uniform milky | 100.30 | 7.62 | 0.210 | 41.3 |
| sterilization | white | | | | |
| Low temperature | Uniform milky | 100.56 | 7.51 | 0.210 | 40.6 |
| 4° C.-5 d | white | | | | |
| High temperature | Uniform milky | 101.03 | 7.48 | 0.211 | 41.2 |
| 40° C.-5 d | white | | | | |
| High temperature | Uniform milky | 100.79 | 7.21 | 0.209 | 40.8 |
| 60° C.-5 d | white | | | | |
| Low temperature | Uniform milky | 99.89 | 7.29 | 0.210 | 39.8 |
| 4° C.-10 d | white | | | | |
| High temperature | Uniform milky | 100.45 | 7.25 | 0.211 | 39.5 |
| 40° C.-10 d | white | | | | |
| High temperature | Uniform milky | 101.25 | 7.12 | 0.213 | 38.5 |
| 60° C.-10 d | white | | | | |
| Low temperature | Uniform milky | 100.52 | 6.98 | 0.210 | 40.2 |
| 4° C.-30 d | white | | | | |
| High temperature | Uniform milky | 100.68 | 6.86 | 0.212 | 40.5 |
| 40° C.-30 d | white | | | | |
| High temperature | Uniform milky | 101.59 | 6.79 | 0.210 | 40.6 |
| 60° C.-30 d | white | | | | |

### Example 30 Sample Batch (0.14%)

| | | | | Particle | Zeta |
|---|---|---|---|---|---|
| | | Content | | size | potential |
| | Appearance | (%) | pH | (μm) | (mV) |
| After | Uniform milky | 101.20 | 7.55 | 0.202 | 42.1 |
| sterilization | white | | | | |
| Low temperature | Uniform milky | 100.20 | 7.48 | 0.202 | 40.2 |
| 4° C.-5 d | white | | | | |
| High temperature | Uniform milky | 99.89 | 7.45 | 0.203 | 40.3 |
| 40° C.-5 d | white | | | | |
| High temperature | Uniform milky | 100.54 | 7.33 | 0.204 | 39.5 |
| 60° C.-5 d | white | | | | |
| Low temperature | Uniform milky | 101.89 | 7.02 | 0.202 | 44.5 |
| 4° C.-10 d | white | | | | |
| High temperature | Uniform milky | 102.35 | 6.85 | 0.202 | 38.5 |
| 40° C.-10 d | white | | | | |
| High temperature | Uniform milky | 100.26 | 6.68 | 0.203 | 40.2 |
| 60° C.-10 d | white | | | | |
| Low temperature | Uniform milky | 100.78 | 6.85 | 0.200 | 40.6 |
| 4° C.-30 d | white | | | | |
| High temperature | Uniform milky | 100.45 | 6.67 | 0.205 | 41.5 |
| 40° C.-30 d | white | | | | |
| High temperature | Uniform milky | 100.58 | 6.69 | 0.208 | 40.6 |
| 60° C.-30 d | white | | | | |

### Example 31 Sample Batch (0.2%)

| | | | | Particle | Zeta |
|---|---|---|---|---|---|
| | | Content | | size | potential |
| | Appearance | (%) | pH | (μm) | (mV) |
| After | Uniform milky | 99.60 | 7.69 | 0.208 | 41.1 |
| sterilization | white | | | | |
| Low temperature | Uniform milky | 99.89 | 7.65 | 0.208 | 41.3 |
| 4° C.-5 d | white | | | | |
| High temperature | Uniform milky | 100.25 | 7.67 | 0.209 | 40.2 |
| 40° C.-5 d | white | | | | |
| High temperature | Uniform milky | 101.26 | 7.46 | 0.210 | 41.5 |
| 60° C.-5 d | white | | | | |
| Low temperature | Uniform milky | 102.21 | 7.99 | 0.207 | 42.1 |
| 4° C.-10 d | white | | | | |
| High temperature | Uniform milky | 100.28 | 7.91 | 0.204 | 40.6 |
| 40° C.-10 d | white | | | | |
| High temperature | Uniform milky | 100.56 | 7.55 | 0.211 | 38.5 |
| 60° C.-10 d | white | | | | |
| Low temperature | Uniform milky | 100.45 | 8.03 | 0.207 | 40.2 |
| 4° C.-30 d | white | | | | |
| High temperature | Uniform milky | 101.02 | 7.68 | 0.209 | 38.5 |
| 40° C.-30 d | white | | | | |
| High temperature | Uniform milky | 99.69 | 7.12 | 0.208 | 39.9 |
| 60° C.-30 d | white | | | | |

### Example 32 Sample Batch (0.4%)

| | | | | Particle | Zeta |
|---|---|---|---|---|---|
| | | Content | | size | potential |
| | Appearance | (%) | pH | (μm) | (mV) |
| After | Uniform | 103.77 | 7.85 | 0.154 | 39.1 |
| sterilization | milky white | | | | |
| Low temperature | Uniform | 103.45 | 7.67 | 0.153 | 43.2 |
| 4° C.-5 d | milky white | | | | |
| High temperature | Uniform | 103.24 | 7.62 | 0.153 | 46.1 |
| 40° C.-5 d | milky white | | | | |
| High temperature | Uniform | 103.12 | 7.48 | 0.154 | 46.3 |
| 60° C.-5 d | milky white | | | | |
| Low temperature | Uniform | 103.62 | 7.31 | 0.154 | 42.9 |
| 4° C.-10 d | milky white | | | | |
| High temperature | Uniform | 103.41 | 7.29 | 0.153 | 42.9 |
| 40° C.-10 d | milky white | | | | |
| High temperature | Uniform | | | | |
| 60° C.-10 d | milky white | 103.24 | 7.08 | 0.153 | 39.6 |
| Low temperature | Uniform | 104.33 | 7.81 | 0.157 | 44.1 |
| 4° C.-30 d | milky white | | | | |
| High temperature | Uniform | 102.67 | 7.51 | 0.157 | 45.9 |
| 40° C.-30 d | milky white | | | | |
| High temperature | Uniform | 100.28 | 7.01 | 0.154 | 43.0 |
| 60° C.-30 d | milky white | | | | |

### Example 33 Sample Batch (0.8%)

| | | | | Particle | Zeta |
|---|---|---|---|---|---|
| | | Content | | size | potential |
| | Appearance | (%) | pH | (μm) | (mV) |
| After | Uniform milky | 99.86 | 7.55 | 0.154 | 48.7 |
| sterilization | white | | | | |
| Low temperature | Uniform milky | 101.36 | 7.51 | 0.153 | 45.6 |
| 4° C.-5 d | white | | | | |
| High temperature | Uniform milky | 100.75 | 7.52 | 0.152 | 44.2 |
| 40° C.-5 d | white | | | | |
| High temperature | Uniform milky | 100.19 | 7.37 | 0.156 | 47.2 |
| 60° C.-5 d | white | | | | |
| Low temperature | Uniform milky | 99.78 | 7.82 | 0.154 | 47.6 |
| 4° C.-10 d | white | | | | |
| High temperature | Uniform milky | 100.73 | 7.68 | 0.154 | 45.6 |
| 40° C.-10 d | white | | | | |
| High temperature | Uniform milky | 100.28 | 7.44 | 0.156 | 45.6 |
| 60° C.-10 d | white | | | | |
| Low temperature | Uniform milky | 101.26 | 7.84 | 0.154 | 49.5 |
| 4° C.-30 d | white | | | | |
| High temperature | Uniform milky | 100.19 | 7.48 | 0.152 | 47.5 |
| 40° C.-30 d | white | | | | |
| High temperature | Uniform milky | 100.29 | 7.00 | 0.152 | 47.2 |
| 60° C.-30 d | white | | | | |

### Example 34 Sample Batch (1.0%)

| | | | | Particle | Zeta |
|---|---|---|---|---|---|
| | | Content | | size | potential |
| | Appearance | (%) | pH | (μm) | (mV) |
| After | Uniform milky | 101.31 | 7.58 | 0.155 | 40.2 |
| sterilization | white | | | | |
| Low temperature | Uniform milky | 100.36 | 7.44 | 0.155 | 42.5 |
| 4° C.-5 d | white | | | | |
| High temperature | Uniform milky | 102.02 | 7.37 | 0.154 | 37.5 |
| 40° C.-5 d | white | | | | |
| High temperature | Uniform milky | 100.56 | 7.28 | 0.155 | 39.8 |
| 60° C.-5 d | white | | | | |
| Low temperature | Uniform milky | 101.25 | 7.78 | 0.158 | 41.5 |
| 4° C.-10 d | white | | | | |
| High temperature | Uniform milky | 100.26 | 7.58 | 0.152 | 40.2 |
| 40° C.-10 d | white | | | | |
| High temperature | Uniform milky | 101.87 | 7.40 | 0.152 | 41.2 |
| 60° C.-10 d | white | | | | |
| Low temperature | Uniform milky | 101.72 | 7.79 | 0.154 | 38.7 |
| 4° C.-30 d | white | | | | |
| High temperature | Uniform milky | 100.69 | 7.52 | 0.156 | 39.6 |
| 40° C.-30 d | white | | | | |
| High temperature | Uniform milky | 100.91 | 7.06 | 0.157 | 40.1 |
| 60° C.-30 d | white | | | | |

6.5 Conclusion: the stability data shows that the indexes of the emulsion sample with sodium glycocholate content of 0.05%-1.0%, such as content, potential, average particle size, and the like, had no significant change compared with 0 when the sample was stored for 30 days, which indicates that the sample was stable.

### 7. Feasibility Investigation of Low pH Sample

Examples 38, 39, and 40 were prepared according to the method of Example 16, using glacial acetic acid to adjust the pH of the primary emulsion and fine-adjusting the content of other formulation components.

### 7.1 Formulation Composition (Strength: 2 mg/mL)

| Formulation | | | | |
|---|---|---|---|---|
| composition | Example 38 | Example 39 | Example 40 | Use |
| Alphaxalone | 2 g | 2 g | 2 g | Active ingredient |
| Soybean oil (for | 100 g | 100 g | 100 g | Oil phase |
| injection) | | | | |
| Medium-chain | 100 g | 100 g | 100 g | Oil phase |
| triglyceride | | | | |
| Egg yolk lecithin | 12 g | 12 g | 12 g | Emulsifier |
| Glycerol | 25 g | 25 g | 25 g | Osmotic pressure |
| | | | | regulator |
| Glycocholic acid | 1 g | 1 g | 1 g | Coemulsifier |
| Sodium hydroxide | 0.085 g | 0.085 g | 0.085 g | pH adjuster |
| Glacial acetic acid | Proper | Proper | Proper | pH adjuster |
| | amount | amount | amount | |
| Sodium oleate | 0.3 g | 0.3 g | 0.3 g | Stabilizer |
| Water for injection | Proper | Proper | Proper | Aqueous |
| | amount | amount | amount | phase |

### 7.2 Results of Sample Detection

| | Example 38 | Example 39 | Example 40 |
|---|---|---|---|
| | Uniform | Uniform | Uniform milky |
| Item | milky | milky | white and slight |
| Appearance | white | white | oil-floating |
| Content (%) | 101.4 | 100.1 | 102.4 |
| pH | 5.66 | 5.14 | 4.59 |
| Particle size (μm) | 0.260 | 0.269 | 0.361 |
| Zeta potential (mV) | 43.1 | 37.1 | 37.9 |

7.3 Conclusion: when the pH of the sample was 5.0 or higher, the indexes such as appearance, content, particle size, potential, and the like were all qualified, and when the pH was less than 5.0, the phenomenon of oil floating appeared, and the particle size was large, which had an unstable trend.

### 7.4 Stability Data

### Example 38

| | | | | Particle | Zeta |
|---|---|---|---|---|---|
| | | Content | | size | potential |
| | Appearance | (%) | pH | (μm) | (mV) |
| After sterilization | Uniform | 101.44 | 5.66 | 0.260 | 43.1 |
| | milky white | | | | |
| High temperature | Uniform | 100.10 | 5.60 | 0.261 | 47.1 |
| 40° C.-10 d | milky white | | | | |
| High temperature | Uniform | 100.33 | 5.52 | 0.261 | 46.5 |
| 60° C.-10 d | milky white | | | | |
| High temperature | Uniform | 100.38 | 5.59 | 0.263 | 46.2 |
| 40° C.-30 d | milky white | | | | |
| High temperature | Uniform | 98.89 | 5.44 | 0.263 | 45.6 |
| 60° C.-30 d | milky white | | | | |

### Example 39

| | | | | Particle | Zeta |
|---|---|---|---|---|---|
| | | Content | | size | potential |
| | Appearance | (%) | pH | (μm) | (mV) |
| After sterilization | Uniform | 100.10 | 5.14 | 0.269 | 37.1 |
| | milky white | | | | |
| High temperature | Uniform | 100.56 | 5.08 | 0.274 | 35.6 |
| 40° C.-5 d | milky white | | | | |
| High temperature | Uniform | 99.12 | 5.08 | 0.275 | 37.6 |
| 60° C.-5 d | milky white | | | | |
| High temperature | Uniform | 98.96 | 5.06 | 0.283 | 36.5 |
| 40° C.-10 d | milky white | | | | |
| High temperature | Uniform | 99.90 | 5.10 | 0.280 | 36.2 |
| 60° C.-10 d | milky white | | | | |
| High temperature | Uniform | 98.12 | 5.09 | 0.279 | 37.5 |
| 40° C.-30 d | milky white | | | | |
| High temperature | Uniform | 98.75 | 5.01 | 0.278 | 36.1 |
| 60° C.-30 d | milky white | | | | |

7.5 Conclusion: when the pH of the sample was higher than 5, the indexes such as appearance, content, Zeta potential, particle size, and the like were qualified under the conditions of storage for 30 days at 40° C. and 60° C., and the decrease of pH was within a controllable range.

### 8. Solubility Test of Alphaxalone in Castor Oil, Long-Chain Oil (Soybean Oil), and Medium-Chain Oil (Medium-Chain Triglyceride)

### 1. Experimental Objective: To Investigate the Solubility of Alphaxalone in Castor Oil

Experimental process: 10 mg of alphaxalone was weighed out, added to 100 g of castor oil at 70° C., and sheared at 10000 rpm for 5 min. The system was then clear. 10 mg of alphaxalone was weighed out again, added to the system, and sheared at 10000 rpm for 3 min. The system was then clear. 30 mg of alphaxalone was weighed out again, added to the system, and sheared at 10000 rpm for 3 min. The system was then clear. Alphaxalone was weighed out several times until the system remained clear when the total amount was 2 g.

Experimental conclusion: the solubility of alphaxalone in castor oil is 2 g or more.

### 2. Experimental Objective: To Investigate the Solubility of Alphaxalone in Soybean Oil

Experimental process: 10 mg of alphaxalone was weighed out, added to 100 g of soybean oil at 70° C., and sheared at 10000 rpm for 5 min. The system was then turbid.

Experimental conclusion: the solubility of alphaxalone in 100 g of soybean oil is less than 10 mg.

### 3. Experimental Objective: To Investigate the Solubility of Alphaxalone in Medium-Chain Oil

Experimental process: 10 mg of alphaxalone was weighed out, added to 100 g of medium-chain oil at 70° C., and sheared at 10000 rpm for 5 min. The system was then clear. 10 mg of alphaxalone was weighed out again, added to the system, and sheared at 10000 rpm for 3 min. The system was then clear. 30 mg of alphaxalone was weighed out again, added to the system, and sheared at 10000 rpm for 3 min. The system was then slightly turbid.

Experimental conclusion: 100 g of medium-chain oil can dissolve 40 mg or less of alphaxalone.

### 9. Summary of Mouse Histamine Release Data

Overall test method: the corresponding solution or formulation was prepared. Administration was performed by intravenous injection, and blood was collected 30 min after administration. After anticoagulation with EDTA, the blood was centrifuged to collect the plasma, and the histamine content in mouse plasma was measured with an ELISA kit.

Blank plasma: blood was directly collected from mice and centrifuged.

Normal saline: normal saline was injected at an administration volume of 5 mL/kg.

Blank fat emulsion (20% medium-long-chain fat emulsion): the administration volume was 5 mL/kg.

Alphaxalone fat emulsion (containing sodium glycocholate, Example 26 sample batch): the dose of the alphaxalone fat emulsion (containing sodium glycocholate) was 7 mg/kg. The ED50 of alphaxalone was about 3.5 mg/kg, thus the dose was 7 mg/kg. The strength of the alphaxalone fat emulsion (containing sodium glycocholate) was 5 mg/mL, which was diluted to 1.4 mg/mL with a blank fat emulsion, and the administration volume was 5 mL/kg.

SBE-β-CD: the preparation method was: 0.7 mL of 13% SBE-β-CD+4.3 mL of normal saline, and the administration volume was 5 mL/kg. (The formulation of the suspension bridge company is alphaxalone dissolved in 13% SBE-β-CD, and the concentration after dissolution is 10 mg/mL, so that this group of experiments referred to the formulation of the suspension bridge company for preparing a drug solution). Cremophor EL: 0.7 mL of Cremophor EL+4.3 mL of normal saline, the concentration of Cremophor EL was 0.14 mL/mL, the dose was 5 mL/kg, and the dose of Cremophor EL was 0.7 mL/kg.

SBE-β-CD & Cremophor EL: 0.7 mL of 13% SBE-β-CD+0.7 mL of Cremophor EL+3.6 mL of normal saline, and the administration volume was 5 mL/kg.

Alphaxalone @ SBE-β-CD & Cremophor EL: 0.7 mL of alphaxalone @ 13% SBE-β-CD (10 mg/mL)+0.7 mL of Cremophor EL+3.6 mL of normal saline, and the administration volume was 5 mL/kg. The final concentration of alphaxalone was 1.4 mg/mL, thus the final dose of alphaxalone was 7 mg/kg, and the dose of Cremophor EL was 0.7 mL/kg.

Alphaxalone @ castor oil (10%) fat emulsion (2 mg/mL): the administration volume was 3.5 mL/kg, and the dose was 7 mg/kg; the administration amount of castor oil was 0.35 mL/kg.

Alphaxalone @ castor oil (7%) fat emulsion (2 mg/mL): this comparative example was prepared by reference to CN94190450.4, Example 1. The administration volume was 3.5 mL/kg, the dose was 7 mg/kg; the administration amount of castor oil was 0.245 mL/kg.

Alphaxalone fat emulsion injection (containing sodium glycocholate data)

| | Plasma histamine |
|---|---|
| | concentration |
| Group | ng/mL |
| Blank plasma | 15.65 ± 2.10 |
| Normal saline | 16.65 ± 3.29 |
| Blank fat emulsion | 15.03 ± 2.18 |
| Alphaxalone fat emulsion | 16.01 ± 3.12 |
| (containing sodium | |
| glycocholate, Example 26) | |
| SBE-β-CD | 15.28 ± 2.33 |
| Cremophor EL | 32.28 ± 1.37 |
| SBE-β-CD&Cremophor EL | 36.82 ± 5.32 |
| Alphaxalone @ SBE-β-CD | 50.40 ± 13.59 |
| & Cremophor EL | |
| Alphaxalone @ castor oil (10%) | 35.32 ± 1.74 |
| fat emulsion (2 mg/mL) | |
| Alphaxalone @ castor oil (7%) | 41.65 ± 5.64 |
| fat emulsion (2 mg/mL) | |

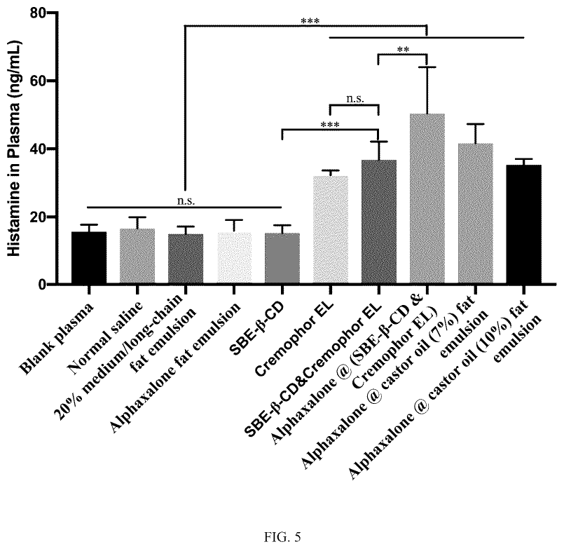

Description of the results: after intravenous injection, for normal saline, blank fat emulsion (20% medium-long-chain fat emulsion), alphaxalone fat emulsion injection (containing sodium glycocholate, Example 26), and sulfobutyl-β-cyclodextrin (SBE-β-CD), mouse plasma histamine concentration had no significant difference from the blank plasma, so that excessive release of histamine in mice was not caused; for Cremophor EL, SBE-β-CD & Cremophor EL, alphaxalone @ (SBE-β-CD & Cremophor EL), alphaxalone @ castor oil (7%) fat emulsion, and alphaxalone @ castor oil (10%) fat emulsion, the mouse plasma histamine concentration was significantly higher than that in the vehicle group, causing excessive release of histamine in mice, which may cause allergic response. The plasma concentration of histamine in mice in the alphaxalone @ (SBE-β-CD & Cremophor EL) group was significantly higher than that of the SBE-β-CD & Cremophor EL group, which indicates that the alphaxalone was dissolved in the SBE-β-CD & Cremophor EL and then promoted the histamine release in the mice, thereby aggravating the allergic degree. The above results show that the alphaxalone formulation containing Cremophor EL and castor oil can cause excessive histamine release in mice after intravenous injection, which may cause allergic response; the alphaxalone fat emulsion injection (containing sodium glycocholate) did not cause allergic response, as shown in FIG. 5.

### 10. Influence of Different Formulations of Alphaxalone on Rat Myoelectricity

Normal saline: normal saline
Blank fat emulsion (normal formulation): the same as Example 1 except that the main drug alphaxalone was not contained
Alphaxalone fat emulsion (2 mg/mL): Example 1
Alphaxalone fat emulsion (10% castor oil) (2 mg/mL): Comparative Example 1
Propofol fat emulsion (10 mg/mL): produced by Jiangsu Nhwa Pharmaceutical Co., Ltd., batch No. BB200215
Blank fat emulsion (containing sodium glycocholate): the same as Example 33 except that the main drug alphaxalone was not contained
Alphaxalone fat emulsion (containing sodium glycocholate) (4 mg/mL alphaxalone): Example 33
Cremophor EL: 1 mL of Cremophor EL+4 mL of normal saline.
SBE-β-CD & Cremophor EL: 1 mL of 13% SBE-β-CD+1 mL of Cremophor EL+3 mL of normal saline.
Alphaxalone @ (SBE-β-CD & Cremophor EL): 1 mL of alphaxalone @ 13% SBE-β-CD (10 mg/mL)+1 mL of Cremophor EL+3 mL of normal saline.

### Test Method:

A 20% urethan solution was injected into the rats intraperitoneally. The anesthetized rats were fixed on an operation plate, the saphenous artery on one side of the hind limb was found and separated, the semitendinosus muscle of the hind limb on the same side was found, and an electrode was inserted. A plastic cannula was inserted into the separated saphenous artery, the drug was injected through the plastic cannula, and the cannula was flushed with normal saline after each injection was completed.

The rat myoelectricity before and after injection was integrated, the ratio of the rat myoelectricity after injection to a baseline was calculated, and the myoelectricity stimulation of the solution to the rat was evaluated.

The injection volume of the drug was 0.1 mL, and the plastic cannula was flushed with normal saline after administration.

### Test Results:

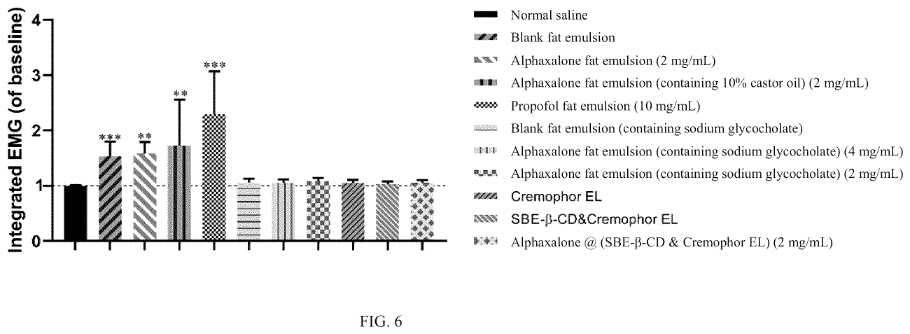

The data show that compared with normal saline, the myoelectricity of rats was remarkably increased after administration of blank fat emulsion, alphaxalone fat emulsion (2 mg/mL, Example 1), alphaxalone fat emulsion (10% castor oil) (2 mg/mL), propofol fat emulsion (manufacturer: Jiangsu Nhwa Pharmaceutical Co., Ltd., batch No. BB200215, 10 mg/mL), and injection irritation and injection pain may be caused in actual use. Blank fat emulsion (containing sodium glycocholate), alphaxalone fat emulsion (containing sodium glycocholate, Example 33) (4 mg/mL), Cremophor EL, SBE-β-CD & Cremophor EL, and alphaxalone @ (SBE-β-CD & Cremophor EL) had no significant effect on rat myoelectricity after administration, which may not cause injection irritation and injection pain, as shown in FIG. 6.

| | Particle | | Integrated EMG | Dunnett's |
|---|---|---|---|---|
| Group | size (μm) | n= | (of baseline) | test |
| Normal saline | / | 19 | 0.99 ± 0.02 | — |
| Blank fat emulsion (common | / | 10 | 1.53 ± 0.26 | ***P < 0.0001 |
| formulation) | | | | |
| Alphaxalone fat emulsion | 0.262 | 4 | 1.58 ± 0.21 | **P = 0.0039 |
| (2 mg/mL) | | | | |
| Alphaxalone fat emulsion (10% | 0.548 | 3 | 1.73 ± 0.83 | **P = 0.0011 |
| castor oil) (2 mg/mL) | | | | |
| Propofol fat emulsion (10 | / | 5 | 2.29 ± 0.77 | ***P < 0.0001 |
| mg/mL) | | | | |
| Blank fat emulsion (containing | 0.155 | 8 | 1.06 ± 0.07 | P = 0.9993 |
| sodium glycocholate) | | | | |
| Alphaxalone fat emulsion | 0.154 | 3 | 1.05 ± 0.07 | P = 0.9996 |
| (containing sodium | | | | |
| glycocholate) (4 mg/mL) | | | | |
| Alphaxalone fat emulsion | / | 3 | 1.09 ± 0.06 | P = 0.9994 |
| (containing sodium | | | | |
| glycocholate) (2 mg/mL) | | | | |
| Cremophor EL | / | 4 | 1.05 ± 0.06 | P = 0.9996 |
| SBE-β-CD&Cremophor EL | / | 4 | 1.03 ± 0.06 | P = 0.9997 |
| Alphaxalone @ (SBE-β-CD & | | 4 | 1.05 ± 0.04 | P = 0.9996 |
| Cremophor EL) | | | | |

Note:
the significance of the Dunnett's test in the table is the result compared with the normal saline group.

1. An alphaxalone fat emulsion injection, comprising alphaxalone or a pharmaceutically acceptable salt thereof, oil for injection, an emulsifier, a coemulsifier, an osmotic pressure regulator, a stabilizer, a pH adjuster, and water for injection; the coemulsifier is a cholic acid compound or a salt thereof.

2. The alphaxalone fat emulsion injection according to claim 1, wherein the oil for injection is soybean oil or a mixture of soybean oil and medium-chain triglyceride.

3. The alphaxalone fat emulsion injection according to claim 2, wherein the weight ratio of the soybean oil to the medium-chain triglyceride in the mixture of the soybean oil and the medium-chain triglyceride is 1-5:1-5, preferably 1:1.

4. The alphaxalone fat emulsion injection according to claim 1, wherein the weight ratio of the alphaxalone or the pharmaceutically acceptable salt thereof to the oil for injection is 1-10:50-300, preferably 1-10:100-300, preferably 1-6:100-300, preferably 1-3:100-300, and preferably 1-3:100-200; or the amount of the oil for injection accounts for 10-30%, preferably 10-20%, of the mass concentration of the fat emulsion injection.

5. The alphaxalone fat emulsion injection according to claim 1, wherein the alphaxalone or the pharmaceutically acceptable salt thereof has a purity greater than 95%.

6. The alphaxalone fat emulsion injection according to claim 1, wherein the emulsifier is egg yolk lecithin, preferably at least one of egg yolk lecithin E-80, egg yolk lecithin PL-100M, and egg yolk lecithin PC-98T, and more preferably a mixture of egg yolk lecithin E-80 and egg yolk lecithin PL-100M in a weight ratio of 1:2; the amount of the pH adjuster is the amount needed to control the pH of the injection to 5.0-8.5;
optionally, the osmotic pressure regulator is glycerol;
optionally, the stabilizer is at least one selected from oleic acid and sodium oleate, preferably sodium oleate;
optionally, the pH adjuster is sodium hydroxide.

7. The alphaxalone fat emulsion injection according to claim 1, wherein the emulsifier is egg yolk lecithin, preferably at least one of egg yolk lecithin E-80, egg yolk lecithin PL-100M, and egg yolk lecithin PC-98T, and more preferably a mixture of egg yolk lecithin E-80 and egg yolk lecithin PL-100M in a weight ratio of 1:2; the amount of the pH adjuster is the amount needed to control the pH of the injection to 6.0-8.5;
optionally, the osmotic pressure regulator is glycerol;
optionally, the stabilizer is at least one selected from oleic acid and sodium oleate, preferably sodium oleate;
optionally, the coemulsifier is a cholic acid compound or a salt thereof;
optionally, the pH adjuster is sodium hydroxide.

8. The alphaxalone fat emulsion injection according to claim 1, wherein the cholic acid compound or the salt thereof is at least one of glycocholic acid, cholic acid, taurocholic acid, glycodeoxycholic acid, taurodeoxycholic acid, chenodeoxycholic acid, glycochenodeoxycholic acid, taurochenodeoxycholic acid, or sodium salts thereof, preferably glycocholic acid and/or sodium glycocholate.

9. The alphaxalone fat emulsion injection according to claim 1, wherein the pH of the injection is 5.0-8.5; the components include the following in parts by weight:
1-10 of alphaxalone;
50-200 of soybean oil;
0-100 of medium-chain triglyceride;
9-15 of emulsifier;
0.1-20 of coemulsifier;
0.1-0.3 of stabilizer;
22-25 of glycerol.

10. The alphaxalone fat emulsion injection according to claim 1, wherein the pH of the injection is 6.0-8.5; the components include the following in parts by weight:
1-10 of alphaxalone;
50-200 of soybean oil;
0-100 of medium-chain triglyceride;
9-15 of emulsifier;
0.1-20 of coemulsifier;
0.1-0.3 of stabilizer;
22-25 of glycerol.

11. The alphaxalone fat emulsion injection according to claim 1, wherein the pH of the injection is 6.0-8.5; the components include the following in parts by weight:
2-8 of alphaxalone;
50-200 of soybean oil;
0-100 of medium-chain triglyceride;
9-12 of emulsifier;
0.1-0 f coemulsifier;
0.1-0.3 of stabilizer;
22-25 of glycerol.

12. The alphaxalone fat emulsion injection according to claim 1, wherein the pH is 6.0-8.5, and the alphaxalone fat emulsion injection comprises per 1 mL:
1-10 mg of alphaxalone;
50-100 mg of soybean oil;
50-100 mg of medium-chain triglyceride;
9-15 mg of egg yolk lecithin;
0.1-10 mg of sodium glycocholate;
0.1-0.3 mg of sodium oleate;
sodium hydroxide in an amount capable of controlling the pH of the injection to 6.0-8.5; and
the balance of water for injection;
or the pH is 6.0-8.5, and the alphaxalone fat emulsion injection comprises per 1 mL:
1-10 mg of alphaxalone;
100-200 mg of soybean oil;
9-15 mg of egg yolk lecithin;
0.1-10 mg of sodium glycocholate;
0.1-0.3 mg of sodium oleate;
sodium hydroxide in an amount capable of controlling the pH of the injection to 6.0-8.5; and
the balance of water for injection.

13. The alphaxalone fat emulsion injection according to claim 1, wherein the pH is 6.0-8.5, and the alphaxalone fat emulsion injection comprises per 1 mL:
1-10 mg of alphaxalone;
50-100 mg of soybean oil;
50-100 mg of medium-chain triglyceride;
9-15 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection;
or the pH is 6.0-8.5, and the alphaxalone fat emulsion injection comprises per 1 mL:
1-10 mg of alphaxalone;
100-200 mg of soybean oil;
9-15 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection.

14. The alphaxalone fat emulsion injection according to claim 1, wherein the pH is 6.0-8.5, and the alphaxalone fat emulsion injection comprises per 1 mL:
1-10 mg of alphaxalone;
100 mg of soybean oil;
100 mg of medium-chain triglyceride;
9-15 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection;
or the pH is 6.0-8.5, and the alphaxalone fat emulsion injection comprises per 1 mL:
1-10 mg of alphaxalone;
200 mg of soybean oil;
9-15 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection.

15. The alphaxalone fat emulsion injection according to claim 1, wherein the pH is 6.0-8.5, and the alphaxalone fat emulsion injection comprises per 1 mL:
2-8 mg of alphaxalone;
100 mg of soybean oil;
100 mg of medium-chain triglyceride;
9-12 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection;
or the pH is 6.0-8.5, and the alphaxalone fat emulsion injection comprises per 1 mL:
2-8 mg of alphaxalone;
200 mg of soybean oil;
9-12 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection;
or the pH is 6.0-8.5, and the alphaxalone fat emulsion injection comprises per 1 mL:
2-8 mg of alphaxalone;
100 mg of soybean oil;
50 mg of medium-chain triglyceride;
9-12 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection;
or the pH is 6.0-8.5, and the alphaxalone fat emulsion injection comprises per 1 mL:
2-8 mg of alphaxalone;
50 mg of soybean oil;
50 mg of medium-chain triglyceride;
9-12 mg of egg yolk lecithin;
0.1-10 mg of sodium glycocholate;
0.1-0.3 mg of sodium oleate;
25 mg of glycerol;
sodium hydroxide in an amount capable of controlling the pH of the injection to 6.0-8.5; and
the balance of water for injection.

16. The alphaxalone fat emulsion injection according to claim 1, wherein the pH is 6.0-8.5, and the alphaxalone fat emulsion injection comprises per 1 mL:
1-10 mg of alphaxalone;
50-200 mg of soybean oil;
50-100 mg of medium-chain triglyceride;
9-15 mg of egg yolk lecithin;
0.1-10 mg of sodium glycocholate;
0.1-0.3 mg of sodium oleate;
22.5-25 mg of glycerol;
sodium hydroxide in an amount capable of controlling the pH of the injection to 6.0-8.5; and
the balance of water for injection;
or the pH is 8.0, and the alphaxalone fat emulsion injection comprises per 1 mL:
2 mg of alphaxalone;
50 mg of soybean oil;
50 mg of medium-chain triglyceride;
4 mg of egg yolk lecithin E-80;
8 mg of egg yolk lecithin PL-100M;
3 mg of sodium glycocholate;
0.3 mg of sodium oleate;
25 mg of glycerol;
sodium hydroxide in an amount capable of controlling the pH of the injection to 8.0; and the balance of water for injection;
or the pH is 6.0, and the alphaxalone fat emulsion injection comprises per 1 mL:
3 mg of alphaxalone;
100 mg of soybean oil;
100 mg of medium-chain triglyceride;
12 mg of egg yolk lecithin E-80;
2 mg of sodium glycocholate;
0.3 mg of sodium oleate;
25 mg of glycerol;
sodium hydroxide in an amount capable of controlling the pH of the injection to 6.0; and the balance of water for injection;
or the pH is 8.0, and the alphaxalone fat emulsion injection comprises per 1 mL:
4 mg of alphaxalone; 50 mg of soybean oil;
50 mg of medium-chain triglyceride;
9 mg of egg yolk lecithin E-80;
4 mg of sodium glycocholate;
0.3 mg of sodium oleate;
25 mg of glycerol;
sodium hydroxide in an amount capable of controlling the pH of the injection to 8.0; and the balance of water for injection;
or the pH is 8.0, and the alphaxalone fat emulsion injection comprises per 1 mL:
4 mg of alphaxalone;
200 mg of soybean oil;
12 mg of egg yolk lecithin E-80;
4 mg of sodium glycocholate;
0.3 mg of sodium oleate;
25 mg of glycerol;
sodium hydroxide in an amount capable of controlling the pH of the injection to 8.0; and the balance of water for injection;
or the pH is 8.0, and the alphaxalone fat emulsion injection comprises per 1 mL:
4 mg of alphaxalone;
200 mg of soybean oil;
12 mg of egg yolk lecithin E-80;
1 mg of sodium glycocholate;
0.3 mg of sodium oleate;
25 mg of glycerol;
sodium hydroxide in an amount capable of controlling the pH of the injection to 8.0; and the balance of water for injection;
or the pH is 8.0, and the alphaxalone fat emulsion injection comprises per 1 mL:
2 mg of alphaxalone;
100 mg of soybean oil;
12 mg of egg yolk lecithin E-80;
4 mg of sodium glycocholate;
0.3 mg of sodium oleate;
25 mg of glycerol;
sodium hydroxide in an amount capable of controlling the pH of the injection to 8.0; and the balance of water for injection;
or the pH is 8.0, and the alphaxalone fat emulsion injection comprises per 1 mL:
4 mg of alphaxalone;
100 mg of soybean oil;
100 mg of medium-chain triglyceride;
15 mg of egg yolk lecithin E-80;
3 mg of sodium glycocholate;
0.3 mg of sodium oleate;
25 mg of glycerol;
sodium hydroxide in an amount capable of controlling the pH of the injection to 8.0; and the balance of water for injection;
or the pH is 8.0, and the alphaxalone fat emulsion injection comprises per 1 mL:
4 mg of alphaxalone;
100 mg of soybean oil;
100 mg of medium-chain triglyceride;
12 mg of egg yolk lecithin E-80;
1 mg of sodium glycocholate;
0.3 mg of sodium oleate;
25 mg of glycerol;
sodium hydroxide in an amount capable of controlling the pH of the injection to 8.0; and the balance of water for injection;
or the pH is 8.0, and the alphaxalone fat emulsion injection comprises per 1 mL:
4 mg of alphaxalone;
100 mg of soybean oil;
100 mg of medium-chain triglyceride;
15 mg of egg yolk lecithin E-80;
3 mg of sodium glycocholate;
0.3 mg of sodium oleate;
25 mg of glycerol;
sodium hydroxide in an amount capable of controlling the pH of the injection to 8.0; and the balance of water for injection;
or the pH is 7.0, and the alphaxalone fat emulsion injection comprises per 1 mL:
5 mg of alphaxalone;
100 mg of soybean oil;
100 mg of medium-chain triglyceride;
15 mg of egg yolk lecithin E-80;
3 mg of sodium glycocholate;
0.3 mg of sodium oleate;
25 mg of glycerol;
sodium hydroxide in an amount capable of controlling the pH of the injection to 7.0; and the balance of water for injection;
or the pH is 7.0, and the alphaxalone fat emulsion injection comprises per 1 mL:
5 mg of alphaxalone;
100 mg of soybean oil;
100 mg of medium-chain triglyceride;
12 mg of egg yolk lecithin E-80;
1 mg of sodium glycocholate;
0.3 mg of sodium oleate;
25 mg of glycerol;
sodium hydroxide in an amount capable of controlling the pH of the injection to 7.0; and the balance of water for injection;
or the pH is 7.5, and the alphaxalone fat emulsion injection comprises per 1 mL:
6 mg of alphaxalone;
50 mg of soybean oil;
50 mg of medium-chain triglyceride;
12 mg of egg yolk lecithin E-80;
2 mg of sodium glycocholate;
0.3 mg of sodium oleate;
25 mg of glycerol;
sodium hydroxide in an amount capable of controlling the pH of the injection to 7.5; and the balance of water for injection;
or the pH is 8.0, and the alphaxalone fat emulsion injection comprises per 1 mL:
7 mg of alphaxalone;
100 mg of soybean oil;
100 mg of medium-chain triglyceride;
12 mg of egg yolk lecithin;
4 mg of sodium glycocholate;
0.3 mg of sodium oleate;
25 mg of glycerol;
sodium hydroxide in an amount capable of controlling the pH of the injection to 8.0; and the balance of water for injection;
or the pH is 7.5, and the alphaxalone fat emulsion injection comprises per 1 mL:
7 mg of alphaxalone;
100 mg of soybean oil;
100 mg of medium-chain triglyceride;
12 mg of egg yolk lecithin;
1 mg of sodium glycocholate;
0.3 mg of sodium oleate;
25 mg of glycerol;
sodium hydroxide in an amount capable of controlling the pH of the injection to 7.5; and the balance of water for injection.

17. A method for preparing an alphaxalone fat emulsion injection, comprising:
(1) heating oil for injection to 50-75° C., adding an emulsifier and alphaxalone, mixing, and shearing until completely dissolved and uniformly dispersed so as to obtain a first mixture containing an alphaxalone oil phase,
wherein preferably, the oil for injection is soybean oil or a mixture of soybean oil and medium-chain triglyceride; more preferably, the oil for injection is a mixture of soybean oil and medium-chain triglyceride in a weight ratio of 1:1;
preferably, the emulsifier is egg yolk lecithin; more preferably, the emulsifier is a mixture of egg yolk lecithin PL-100M and egg yolk lecithin E-80;
(2) heating water for injection to 50-75° C., then adding a coemulsifier, a stabilizer, and an osmotic pressure regulator, and stirring until uniformly mixed so as to obtain a second mixture forming an aqueous phase, preferably, the coemulsifier is sodium glycocholate, the stabilizer is sodium oleate, and the osmotic pressure regulator is glycerol;
(3) mixing the first mixture and the second mixture and shearing during the mixing process so as to obtain a third mixture forming primary emulsion;
(4) adjusting the pH of the third mixture using a pH adjuster so as to obtain a fourth mixture, and performing low-pressure homogenization once to obtain a fifth mixture, preferably, the pH adjuster is sodium hydroxide;
(5) homogenizing the fifth mixture at high pressure 3 times or more, preferably 4-8 times, and more preferably 5-6 times, with nitrogen introduced below the liquid surface to saturation during the homogenization process so as to obtain a sixth mixture;
(6) filling the sixth mixture into a container, introducing nitrogen for protection, and performing wet-heat sterilization so as to obtain an alphaxalone fat emulsion injection with a pH of 5.0-8.5; preferably, the pH is 6.0-8.5.

18. A method for preparing an alphaxalone fat emulsion injection, comprising:
(1) heating oil for injection to 50-75° C., then adding an emulsifier and alphaxalone, mixing, and shearing until completely dissolved and uniformly dispersed so as to obtain a first mixture containing an alphaxalone oil phase,
preferably, the oil for injection is soybean oil or a mixture of soybean oil and medium-chain triglyceride; more preferably, the oil for injection is a mixture of soybean oil and medium-chain triglyceride in a weight ratio of 1:1;
preferably, the emulsifier is egg yolk lecithin; more preferably, the emulsifier is a mixture of egg yolk lecithin PL-100M and egg yolk lecithin E-80;
(2) heating water for injection to 50-75° C., then adding a coemulsifier, a stabilizer, an osmotic pressure regulator, and a pH adjuster, and stirring uniformly so as to obtain a second mixture forming an aqueous phase, preferably, the coemulsifier is sodium glycocholate, the stabilizer is sodium oleate, the osmotic pressure regulator is glycerol, and the pH adjuster is sodium hydroxide;
(3) mixing the first mixture and the second mixture and shearing during the mixing process so as to obtain a third mixture forming primary emulsion;
(4) optionally, adjusting the pH of the third mixture using a pH adjuster so as to obtain a fourth mixture, wherein preferably, the pH adjuster is sodium hydroxide;
(5) homogenizing the mixture obtained in step (3) or (4) at low pressure once to obtain a fifth mixture; homogenizing the fifth mixture at high pressure 3 times or more, preferably 4-8 times, and more preferably 5-6 times, with nitrogen introduced below the liquid surface to saturation during the homogenization process so as to obtain a sixth mixture;
(6) filling the sixth mixture into a container, introducing nitrogen for protection, and performing wet-heat sterilization so as to obtain an alphaxalone fat emulsion injection with a pH of 5.0-8.5; preferably, the pH is 6.0-8.5.

19. The method according to claim 17, wherein the shearing and mixing are performed at a rotation speed of 5000-16000 rpm for 5-25 min; preferably, the rotation speed of the shearing during the mixing process is 10000 rpm, and the rotation speed of the shearing after the completion of mixing is 15000-16000 rpm.

20. The method according to claim 18, wherein the shearing and mixing are performed at a rotation speed of 5000-16000 rpm for 5-25 min; preferably, the rotation speed of the shearing during the mixing process is 10000 rpm, and the rotation speed of the shearing after the completion of mixing is 15000-16000 rpm.
